(19) United States
(12) Patent Application Publication
(10) Pub. No.: US 2025/0268620 A1
(43) Pub. Date: Aug. 28, 2025

(54) Atherectomy Catheter

(71) Applicant: Center for Heart and Circulation PA, Leesburg, FL (US)

(72) Inventor: Vishnu Yelamanchi, Leesburg, FL (US)

(73) Assignee: Center for Heart and Circulation PA, Leesburg, FL (US)

(21) Appl. No.: 19/062,312

(22) Filed: Feb. 25, 2025

Related U.S. Application Data
(60) Provisional application No. 63/557,591, filed on Feb. 25, 2024.

(51) Int. Cl. A61B 17/3207; A61B 17/22; A61B 17/32

(52) U.S. Cl. CPC A61B 17/320725; A61B 17/22004; A61B 2017/320052; A61B 2017/320064; A61B 2017/320716; A61B 2217/005; A61B 2217/007

(57) ABSTRACT

Implemented is a catheter for removing material in a body lumen. The catheter includes a catheter tube, a flexible drive shaft, an infusion lumen, an extraction lumen, and a head configured to remove the material in the body lumen. The head includes a housing connected to the catheter tube. The housing includes an infusion aperture fluidly coupled to the infusion lumen, an extraction aperture fluidly coupled to the extraction lumen, and a cutting chamber having a cutout therein. The head further includes a balloon and a probe connected to and driven by the flexible drive shaft. The probe is disposed within the cutting chamber, and the probe is configured to remove the material from the body lumen, at the cutout, and pulverize the material within the cutting chamber.

### CROSS-REFERENCE TO RELATED APPLICATIONS

This application claims priority and hereby incorporates by reference in its entirety U.S. Provisional Patent Application Ser. No. 63/557,591 entitled “INTERVENTIONAL CATHETER SYSTEM FOR PLAQUE PULVERIZATION, CUTTING AND REMOVAL. MULTIFUNCTION ATHERECTOMY CATHETER” and filed on Feb. 25, 2024, the entire contents of which are hereby incorporated herein by reference.

### BACKGROUND

In endovascular surgeries, various techniques with corresponding interventional tools exist for removing atheroma, e.g., plaque, lesions, or other fatty material, from arterial walls. In a balloon angioplasty, a balloon is inserted into the subject artery and inflated at its stenotic region, typically at 5-20 atmospheres, to displace material and restore the lumen. However, a balloon cannot fragment the material to a sufficiently small size, such as 6-8 microns, nor remove debris from the lumen, which may lead to a suboptimal expansion of the lumen, a recoiling of material back into the lumen, or a worse clinical outcome. A balloon expandable or self-expandable stent may be placed at the site of the lesion as a scaffolding. An eccentric, large, and hard mineralized (calcified) lesion often limits the expansion of the stent to its intended size, leading to negative clinical outcomes. In an atherectomy, a surgeon typically uses an atherectomy catheter that has a rotatable blade, a burr, a crown, or a laser emitter for forcibly cutting and removing the material. Other catheters may include acoustic emitters or fluid sprayers, such as a pulsatile acoustic pressure wave emitter for fragmenting and modifying the large calcified lesions. Given the abrasive nature of atherectomy catheters such devices can damage arterial walls, causing vessel trauma or rupture, when attempting to remove material therefrom. Therefore, such atherectomy catheters are not used in internal, non-compressible parts of the body.

### SUMMARY

In one aspect, there is provided an atherectomy catheter including a multipurpose head or operational hub for targeting a wide variety of material and/or specific types of material, e.g., lesions, in a vessel. The head includes a housing with a cutting chamber, an operational probe rotatable and translatable within the housing for shear cutting material by rotating and/or breaking up material by oscillating within the bounds of the cutting chamber. The head also includes a balloon located on the housing, opposite of the cutting chamber, which pushes the houses toward the material. The head may also include integrated features for the irrigation and aspiration of the cutting chamber. In an example, the head can be configured for the standalone irrigation and/or aspiration of a clot, loose plaque debris, or other foreign body in the body lumen, outside of the cutting chamber. In another example, the probe can perform two functions for linear vibration impact for plaque pulverization at an oscillation rate (vibrating and thus resonating at a natural frequency of the plaque, primarily for deforming the plaque) and rotational cutting for shear cutting the plaque from the wall (only cutting the plaque within the cutting chamber to reduce or substantially eliminate damage of the body lumen).

In comparison to known atherectomy catheters, the catheter disclosed herein includes integrated safeguards for reducing damage to the vessel. Additionally, the catheter may quickly and efficiently remove material from the body lumen by cutting with the rotating bladed probe, angioplasty with the integrated balloon, and/or vibro-impact resonant frequencies using the probe as an oscillating impactor (delivering resonant frequency energy causing material deformation and disintegration). Thereby, the catheter can efficiently remove material using multiple different modalities and may target a specific type of material via resonant frequency.

This Summary is provided to introduce a selection of concepts in a simplified form that is further described below in the Detailed Description. This Summary is not intended to identify key features or essential features of the claimed subject matter, nor is it intended to be used as an aid in determining the scope of the claimed subject matter. Furthermore, the claimed subject matter is not limited to implementations that solve any or all disadvantages noted in any part of this disclosure. It will be appreciated that the above-described subject matter may be implemented as a computer-controlled apparatus, a computer process, a computing system, or as an article of manufacture such as one or more computer-readable storage media. These and various other features will be apparent from reading the following Detailed Description and reviewing the associated drawings.

### DESCRIPTION OF THE DRAWINGS

FIG. 1 shows an illustrative representation of a catheter system which includes a catheter for removing material from a body lumen;

FIG. 2 shows an illustrative representation of the catheter system of FIG. 1 which generally includes a drive, a control unit, a roller pump, and a power source;

FIG. 3 shows an illustrative representation of an exploded view of the catheter of FIG. 1 which generally includes a catheter tube, a flexible drive shaft, and a head including a housing, a probe disposed within the housing, a balloon connected to the housing, and a nose connected to the housing;

FIG. 4 shows an illustrative representation of the catheter in a retracted position, wherein the probe is retracted, at least partially exposing a cutting chamber of the housing;

FIG. 5 shows an illustrative representation of the catheter in an extended position, wherein the probe is extended, located distally closer to the nose, and seated within the cutting chamber of the housing;

FIG. 6 shows an illustrative representation of the catheter in operation, wherein the catheter is in the process of being located next to material, i.e., plaque, in a body lumen, with the probe retracted;

FIG. 7 shows an illustrative representation of the catheter in operation, wherein the catheter is located in a desired position within the body lumen, and the probe is extended and actively removing the material from the body lumen;

FIG. 8 shows an illustrative representation of a side view of the catheter;

FIG. 9 shows an illustrative representation of a cross-sectional view of the catheter, taken across line 9-9 in FIG. 8;

FIG. 10 shows an illustrative representation of another side view of the catheter;

FIG. 11 shows an illustrative representation of a cross-sectional view of the catheter, taken across line 11-11 in FIG. 10;

FIG. 12 shows an illustrative representation of an end perspective view of the catheter tube which includes a curved and ellipsoidal infusion lumen and an annular extraction lumen;

FIG. 13 shows an illustrative representation of a side perspective view of the nose of the catheter;

FIG. 14 shows an illustrative representation of a cross-sectional view of the nose, taken across a longitudinal axis of the nose;

FIG. 15 shows an illustrative representation of an elevated side perspective view of the head of the catheter of FIG. 1;

FIG. 16 shows an illustrative representation of an end view of a proximal end of the assembled head of the catheter of FIG. 1;

FIG. 17 shows an illustrative representation of a side view of the housing in isolation;

FIG. 18 shows an illustrative representation of a side perspective view of the housing, illustrating the cutting chamber;

FIG. 19 shows an illustrative representation of another side perspective view of the housing, illustrating a balloon compartment opposite the cutting chamber;

FIG. 20 shows an illustrative representation of a cross-sectional view of the housing, taken across a longitudinal axis of the housing;

FIG. 21 shows an illustrative representation of an elevated side perspective view of the probe of the catheter of FIG. 1 in isolation;

FIG. 22 shows an illustrative representation of an end perspective view of a proximal end of the probe of FIG. 21;

FIG. 23 shows an illustrative representation of a side view of the probe of FIG. 21, illustrating the annular side wall and one of the cutting blades of the probe;

FIG. 24 shows an illustrative representation of another side view of the probe of FIG. 21, illustrating both of the cutting blades of the probe;

FIG. 25 shows an illustrative representation of an elevated side perspective view of another embodiment of a probe which includes dual sided blades with a total of four cutting blades;

FIG. 26 shows an illustrative representation of a side view of the probe of FIG. 25;

FIG. 27 shows an illustrative representation of an elevated side perspective view of another embodiment of a catheter which includes a bladeless probe;

FIG. 28 shows an illustrative representation of a side view of the catheter of FIG. 27;

FIG. 29 shows an illustrative representation of an end view of the catheter tube of the catheter of FIG. 27;

FIG. 30 shows an illustrative representation of an elevated side perspective view of the assembled head of the catheter of FIG. 27;

FIG. 31 shows an illustrative representation of an end view of a distal end of the assembled head of the catheter of FIG. 27, illustrating the nozzle channels and the nozzles of the housing;

FIG. 32 shows an illustrative representation of an end view of a proximal end of the assembled head of the catheter of FIG. 27, illustrating the infusion lumen, the extraction lumen, and the nozzles of the head;

FIG. 33 shows an illustrative representation of an elevated side perspective view of the housing of the catheter of FIG. 27 in isolation;

FIG. 34 shows an illustrative representation of a side view of the housing of FIG. 33;

FIG. 35 shows an illustrative representation of a top view of the housing of FIG. 33;

FIG. 36 shows an illustrative representation of a cross-sectional view of the housing, taken across line 36-36 of FIG. 35;

FIG. 37 shows an illustrative representation of an elevated side perspective view of the probe of the catheter of FIG. 27 in isolation;

FIG. 38 shows an illustrative representation of an elevated side perspective view of another embodiment of a probe in isolation;

FIG. 39 shows an illustrative representation of an end view of a proximal end of the probe of FIG. 38;

FIG. 40 shows an illustrative representation of a perspective and exploded view of another embodiment of a catheter which generally includes a catheter tube and a head with a tubular housing, bladed probe, and a nose;

FIG. 41 shows an illustrative representation of a perspective view of the tubular housing of the catheter of FIG. 40 in isolation, illustrating a proximal end and a central post extending upwardly from a ledge thereof;

FIG. 42 shows an illustrative representation of another perspective view of the tubular housing of FIG. 41, illustrating a distal end thereof;

FIG. 43 shows an illustrative representation of a perspective view of the bladed probe of the catheter of FIG. 40 in isolation, illustrating a proximal end thereof; and

FIG. 44 shows an illustrative representation of another perspective view of the bladed probe of FIG. 43, illustrating a distal end thereof.

Like reference numerals indicate like elements in the drawings. Elements are not drawn to scale unless otherwise indicated.

### DETAILED DESCRIPTION

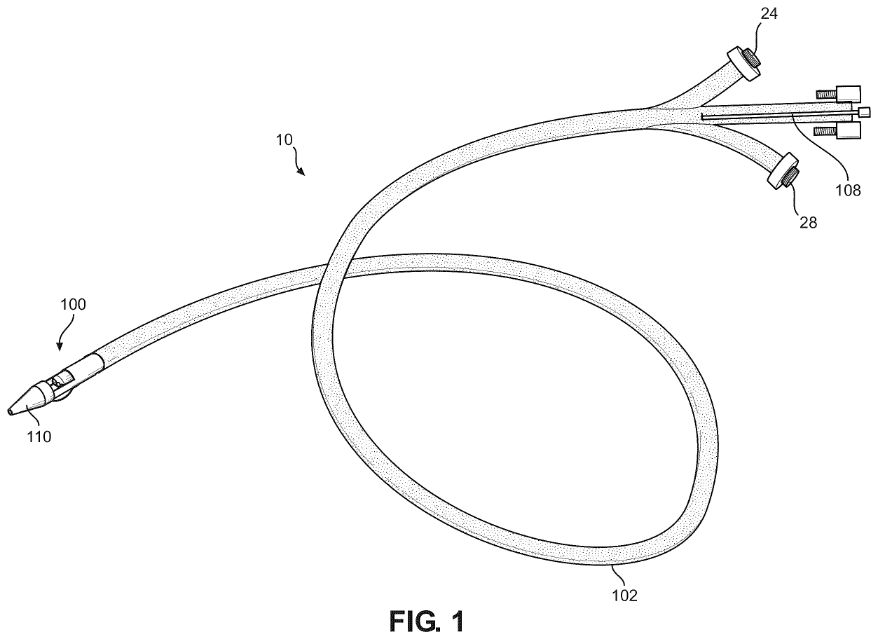

FIGS. 1-3 show an illustrative representation of an exemplary embodiment of a catheter system 10 for removing unwanted material, such as plaque, lesions, fatty deposits, or other material, from an artery, vein, or other body lumen. The catheter system 10 generally includes an atherectomy catheter 100 (athero-thrombectomy catheter), a drive 12 for moving the catheter 100 (e.g., linearly translating and/or rotating the catheter 100), a roller pump 14 for supplying and extracting fluid to and from the catheter 100, a control unit 16, and a power source 18 (e.g., a 120V wall outlet) for supplying electrical power to the drive 12, the roller pump 14, and the control unit 16. The catheter 100 generally includes a catheter tube or main lumen 102 (e.g., at least a dual-lumen tube) with an infusion (i.e., inflow) lumen 104 and an extraction (i.e., outflow) lumen 106 disposed therein, a flexible drive shaft 108 disposed within the catheter tube 102 (e.g., within the extraction lumen 106) driven by the drive 12, a head or operational hub 110 connected to the catheter tube 102 and the flexible drive shaft 108, and a guide wire 112 extending through the catheter tube 102 and the head 110 for guiding the head 110 within the body lumen.

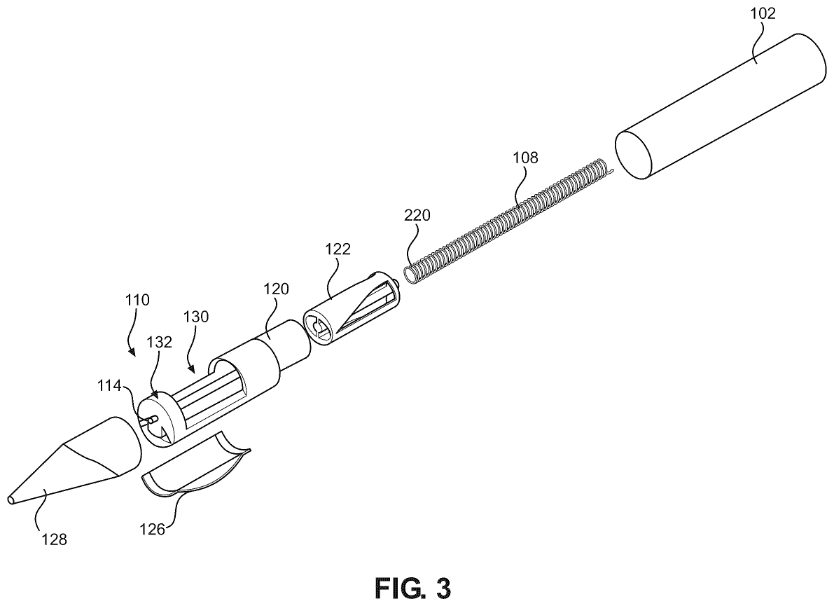

The head 110 of the catheter 100 is configured to perform multiple differing functions to efficiently and safely remove material from the body lumen. Referring specifically to FIG. 3, the head 110 includes a housing 120, an operational probe 122, with one or more blades 124, that is fully disposed within the housing 120, a balloon 126 at least partially disposed within the housing 120, and a nose 128 connected to the housing 120. The probe 122 is connected to and driven by the flexible drive shaft 108, and the probe 122 is disposed within a cutting chamber or probe compartment 130 of the housing 120. The probe 122 is configured to cut and/or pulverize material in conjunction with the cutting chamber 130, as discussed further herein. In one embodiment, the guide wire 112 may be disposed within a guide wire tube 114 (FIG. 3) which extends through the head 110. In an example, the guide wire 112 is disposed within a series of designated guide wire lumens in the drive 12, the flexible drive shaft 108 (disposed within the extraction lumen 106 of the catheter tube 102), the probe 122, and the nose 128 for guiding the head 110 within the body lumen. The guide wire tube 114 may also help stabilize the probe 122 as the probe 122 rotates and translates (e.g., axially vibrates) within the housing 120.

The catheter 100 can effectively and safely remove multiple differing kinds of plaque without damaging the body lumen. In particular, the catheter 100 can stabilize itself via the balloon 126, shear cut material via the probe 122 and cutting chamber 130, remove material from the body lumen and/or pulverize hard calcified plaque to a deformable or excisable material via oscillation impact against the cutting chamber 130, irrigate the cutting chamber 130 (reversing fluid flow to eject the material fragments out of the cutting chamber 130), aspirate (i.e., suck in) fluid, blood, and/or material in an area around the cutting chamber 130 (and the area within the cutting chamber 130) via generating a faster fluid flow through the cutting chamber 130, which according to Bernoulli's principle creates a pressure differential between a relatively high pressure area in the body lumen and a lower pressure area in the cutting chamber 130 of the housing 120, created by the faster fluid flow therein. Furthermore, the head 110 of the catheter 100 can subsequently extract the material fragments out through the cutting chamber 130 and into the extraction lumen 106. In another example, the probe 122 can pulverize cut material within the cutting chamber 130 into smaller pieces or fragments. In comparison to a prior art atherectomy catheter, such as a burr, crown, blade or laser emitter, which may scrape, ablate, cut, or otherwise damage an arterial wall by nature of being freely exposed, the catheter 100 has a built-in or integrated safeguard via housing 120 the probe 122 entirely within the housing 120, preventing the probe 122 from directly contacting the body lumen. Since the probe 122 of the catheter 100 remains within the housing 120 at all times during operation thereof, the catheter 100 only removes material that is located within the bounds of the housing 120 via shearing (i.e., shear cutting) the material between the cutting chamber 130 and the probe 122. Additionally, in one embodiment, the catheter 100 may remove plaque via pulsating at a resonant frequency that causes the material to accordingly vibrate, which in turn causes internal stress (e.g., tensile, compressive, shear stress, and/or strain) that breaks up or weakens the material for subsequent cutting, pulverization, and/or balloon (and/or stent) expansion. Furthermore, in one embodiment, the inflation of the balloon 126 can assist in locating and maintaining a position of the head 110, compressing and stabilizing the cutting chamber 130 against the material and/or body lumen. Still further, the simultaneous irrigation and extraction of material fragments from the cutting chamber 130 can help to remove dislodged material from the body lumen, significantly reducing the risk of debris in the body lumen, causing downstream obstruction. Therefore, the catheter 100 reduces complications from debris and the risk of vessel trauma or perforation, particularly in non-compressible vascular areas within the body.

The drive 12 of the catheter system 10 may comprise one or more electric motors for linearly moving (e.g., oscillating or vibrating) the flexible drive shaft 108 along the longitudinal axis of the catheter tube 102 and/or rotating the flexible drive shaft 108 clockwise and/or counter clockwise. The electric motor can be any desired commercially available electric motor. In one embodiment, the drive 12 can be a handheld drive unit which attaches directly to the flexible drive shaft 108. The flexible drive shaft 108 extends through the catheter tube 102. The flexible drive shaft 108 may be disposed within a designated shaft lumen, a guide wire lumen, or within the extraction lumen 106. In an example, the flexible drive shaft 108 is disposed within the extraction lumen 106. If equipped with a designated shaft lumen, the catheter 100 may further include a lubrication port and accompanying line or channel fluidly coupled in between the fluid source and the shaft lumen for providing fluid, e.g., saline or other lubrication, to the flexible drive shaft 108. The lubrication fluid can be siphoned from the same fluid source as the infusion lumen 104 or may be provided by a separate fluid source. The flexible drive shaft 108 may comprise any desired shaft, such as a coiled, braided, or reinforced shaft or tube.

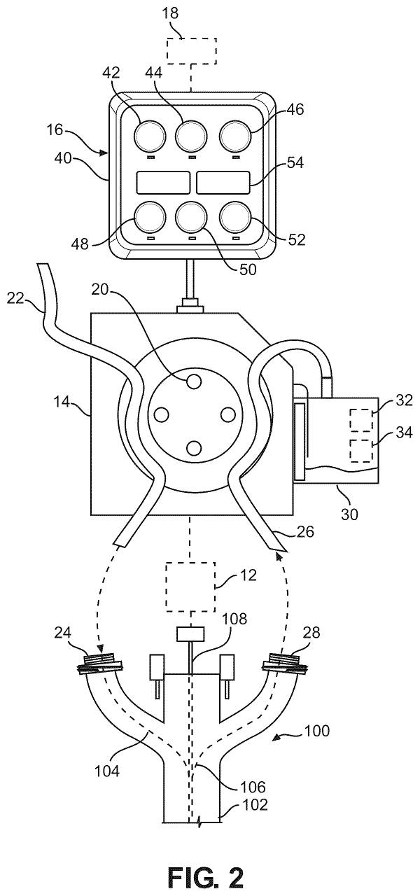

Referring specifically to FIG. 2, the roller pump 14 can comprise a continuous flow roller pump 14 which is driven by an electric motor (not shown) and radially disposed rollers 20. The roller pump 14 operates as a positive displacement pump with an adjustable speed for pumping fluid, e.g., saline or other fluid mixture, from a fluid bag (not shown) and through an infusion tube 22 connected to an infusion port 24 of the catheter tube 102 that feeds into the infusion lumen 104, and suctioning the fluid out of the extraction lumen 106 via an extraction tube 26 connected to an extraction port 28 of the catheter tube 102. The roller pump 14 deposits the extracted fluid, e.g., a combination of material fragments, blood, saline, etc., into a waste or collection tank 30 (FIG. 2). In operation, the roller pump 14 can operate the infusion and extraction fluid flow simultaneously or independently of one another. For example, in one embodiment, the roller pump 14 can respectively pump and extract fluid to and from the infusion and extraction lumens 104, 106 together in tandem to simultaneously perform the irrigation and extraction functions of the catheter 100. Also, for example, the roller pump 14 can extract fluid through the extraction lumen 106, alone in isolation, to perform the aspiration function of the catheter 100.

In one embodiment, the roller pump 14 may additionally include an analysis device 32, such as a cell counter or other blood component analysis device, for automatically measuring one or more characteristics of the extracted fluid (FIG. 2). For example, the cell counter 32 may measure the value and/or amount of components, e.g., cells, of the whole blood within the extraction lumen 106 and/or the collection tank 30. In another example, the cell counter 32 may measure the components of the whole blood as a control sample, drawn directly from the body lumen. Furthermore, a dilution ratio may also be automatically calculated based on the periodic measurements of cell count or other blood component concentrations in the material (e.g., aspirate) from the collection tank 30, and/or test sample, via various point-of-care testing methods. The analysis device may comprise any desired cell counting machine, such as an image-based cell counter, a flow cytometer, or an impedance-based counter (e.g., a Coulter counter), or an i-STAT machine, as non-limiting examples. In one embodiment, the roller pump 14 may also include one or more sensors 34 for measuring fluid flow and/or an amount of collected waste in the collection tank 30 (FIG. 2). In an example, a collection sensor 34 may be connected to the collection tank 30 for measuring the amount of collected waste. The collection sensor 34 may comprise an optical, pressure, or buoyancy sensor. Using the sensed amount of waste, the catheter system 10 may also determine an estimated value of the amount of blood loss in real time, as a function of the dilution ratio (control sample to test sample) and the sensed volume aspirated into the collection tank 30, at periodic time intervals or at predetermined collected volume increments (e.g., 100 ml).

The control unit 16 (or panel) is operably connected to the drive 12 and the roller pump 14. The control unit 16 can include a computer, with a memory and a hardware processor, a display screen, and one or more handheld and/or foot operated devices for controlling the various functions of the drive 12 and roller pump 14. The control unit 16 can comprise a single device or multiple devices operably connected to one another via a network. The device(s) can include human operable buttons including, depressible mechanical buttons, switches, dials, foot pedals, a display screen with various manipulable icons, or other user input devices such that the various operations of the catheter system 10 can be manipulated by one or more users via their hands, feet, and/or voice command. In one embodiment, referring specifically to FIG. 2, the control unit 16 may comprise an interface device 40 that includes an on/off switch 42, a pump activation and speed control button 44 for irrigation, aspiration, and extraction, a motor activation and speed button 46 for rotating the probe 122 clockwise or counter clockwise and/or advance and withdraw the head 110, a pulsation or oscillation button 48 for controlling a linear movement of the probe 122 which determines the resonant frequency at which the probe 122 vibrates, a probe position button 50 for controlling a position of the probe 122 relative to the housing 120, i.e., for positioning the probe 122 in its extended or retracted positions, a balloon inflation and deflation button 52 for independently activating one or more balloons 126, and a display screen 54 for displaying and/or allowing the control of various parameters of the catheter system 10, including fluid flow rate, position of the probe 122, a frequency of the probe 122, a real-time and sensed position of the probe 122 via an optical sensor (not shown), a cell count, or any other desired information. In one embodiment, the control unit 16 can automatically determine the cell count, a blood loss volume, a fluid flow rate in the infusion lumen 104, the head 110, or the extraction lumen 106, an approximate frequency of the probe 122 based on oscillations thereof within a preset time period, or any other desired parameter of the catheter system 10.

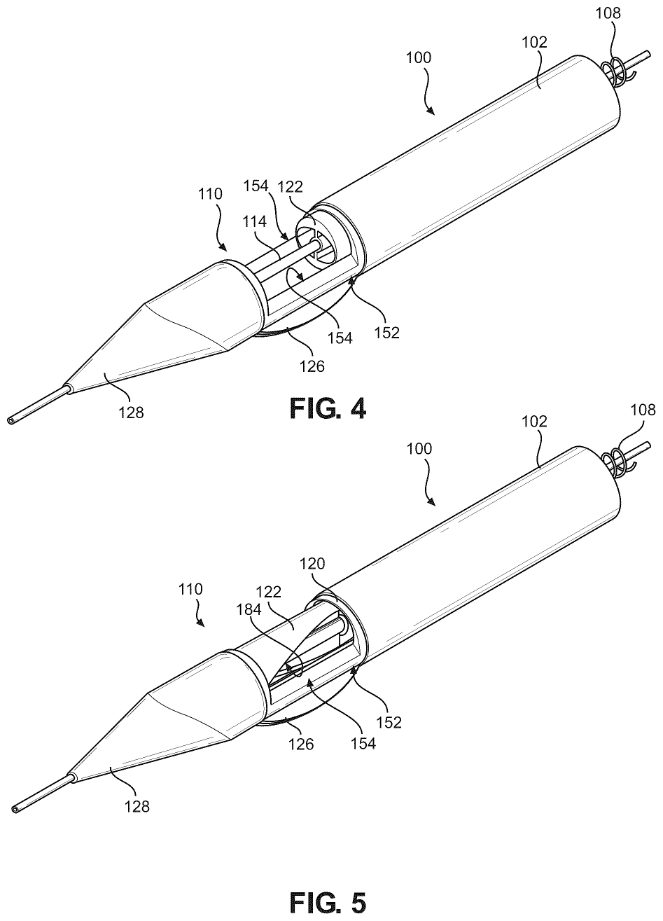

Referring to FIGS. 4-7, the probe 122 can be translatable in between various positions to accommodate certain procedures or functions of the catheter 100. For example, in one embodiment, the probe 122 can be linearly translated between a retracted or transport position (FIG. 4) wherein the probe 122 is substantially housed within the housing 120 such that the blade(s) thereof is (are) covered by the housing 120 to minimize damage during positioning of the catheter 100. Also, the probe 122 can be rotated into an inactive position, wherein the blades 124 of the probe 122 are hidden (facing inwardly toward the cutting chamber 130). In other words, the probe 122 can be positioned in a transport or blade-hidden position, wherein the blades 124 are substantially covered and hidden by the housing 120, e.g., the interior wall of the cutting chamber 130. Therein, during insertion or removal of the head 110, the bladed probe 122 can be protected within the housing 120, which in turn protects the body lumen, preventing damage to the body lumen. In the retracted position, the balloon 126 can be deflated, decreasing the overall profile of the catheter 100. Furthermore, minimal to no fluid may be supplied to the catheter 100 by the roller pump 14 during the positioning or removal of the catheter 100.

Additionally, for example, the probe 122 can be translated distally (closer to the nose 128) into an extended or operational position (FIG. 5), wherein the probe 122 is substantially exposed and fully seated within the cutting chamber 130. In the extended position, the probe 122 is ready to begin rotating for cutting material from the body lumen and/or oscillating for deforming, dislodging, and/or pulverizing the material. In the extended position, the probe 122 is located distally closer to the nose 128 than in the retracted position.

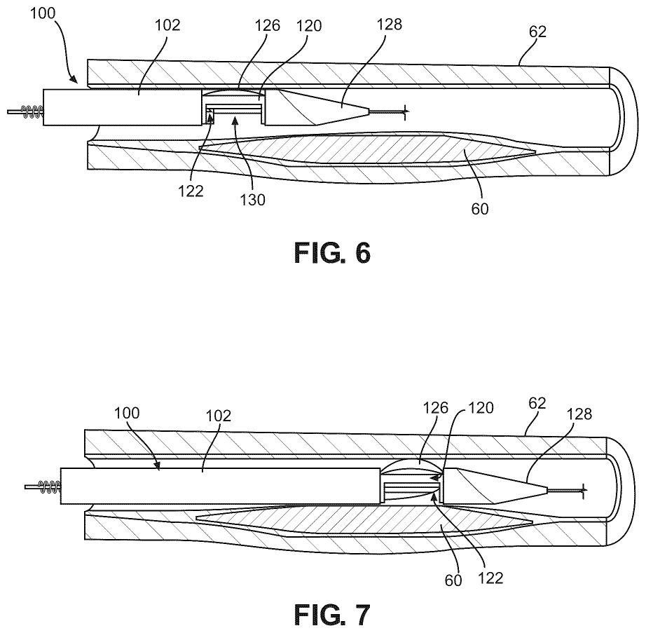

FIG. 6 illustrates the catheter 100 as the catheter 100 is being located next to the material, e.g., lesion 60, at a desired target location within the body lumen, e.g., vessel 62. In an example, the catheter 100 can be delivered and removed through a sheath over the guide wire 112 to the desired target location of the lesion 60 inside of the vessel 62. When inserting the catheter 100 into the vessel 62, the probe 122 may first be moved into its retracted position, as shown in FIG. 6, so that the probe 122 does not undesirably cut or otherwise damage the body lumen whilst moving the catheter 100 through the body lumen to a desired location. The user can advance, withdraw, and/or rotate the catheter 100 manually or by manipulating the drive 12.

FIG. 7 illustrates the catheter 100 in an exemplary operation, wherein the catheter 100 is presently removing the lesion 60 from the vessel 62. In an example, the head 110 can be initially advanced beyond the lesion 60. The probe 122 can be moved into its extended position such that the probe 122 is seated within the cutting chamber 130. Thereafter, the probe 122 can be rotated, and/or oscillated, and the head 110 can be slowly withdrawn longitudinally across the lesion 60, removing portions of the material and/or pulverizing the material along its length. In an example, once a pass is completed, the direction of rotation of the probe 122 can be reversed and the head 110 can be slowly advanced forwardly across the lesion 60. This excision process can be repeated by alternating the direction of blade rotation with each rearward and forward cycle, respectively. In another example, once a pass is completed, the head 110 can be readvanced beyond the lesion 60 and the direction of rotation of the probe 122 may remain the same and/or be reversed for repeated cutting and/or pulverization. The excision process can include alternating forward and rearward movement cycles and/or a stationary cycle wherein the head 110 concentrates on a specific, localized area of the lesion 60. In one embodiment, due to the orientation of the blades 124 of the probe 122, rotational plaque excision can be performed in a forward, rearward, and/or stationary cycle (or fashion) of the head 110. In one embodiment, the oscillation impact pulverization can be performed in a forward, rearward, and/or stationary cycle of the head 110. After completing one radial section of the material 60, the device is rotated directionally to target a different radial section of the material 60. In one example, the atherectomy cycle is then repeated by sliding the catheter 100 longitudinally (e.g., back and forth) along the length of the material 60. In this regard, the cutting procedure of the catheter 100 may function somewhat similarly to a carpenter's hand planer for shaving off sequential layers of material, until the catheter 100 restores the healthy profile of the vessel 62.

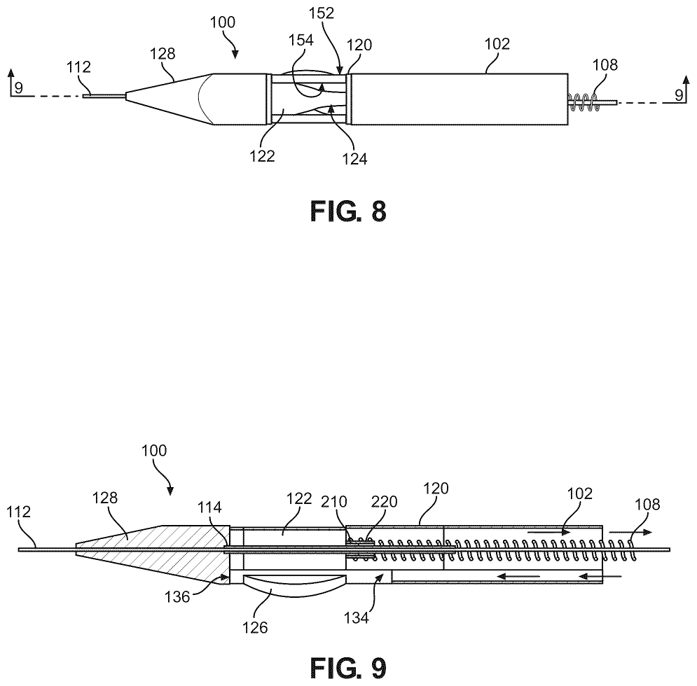

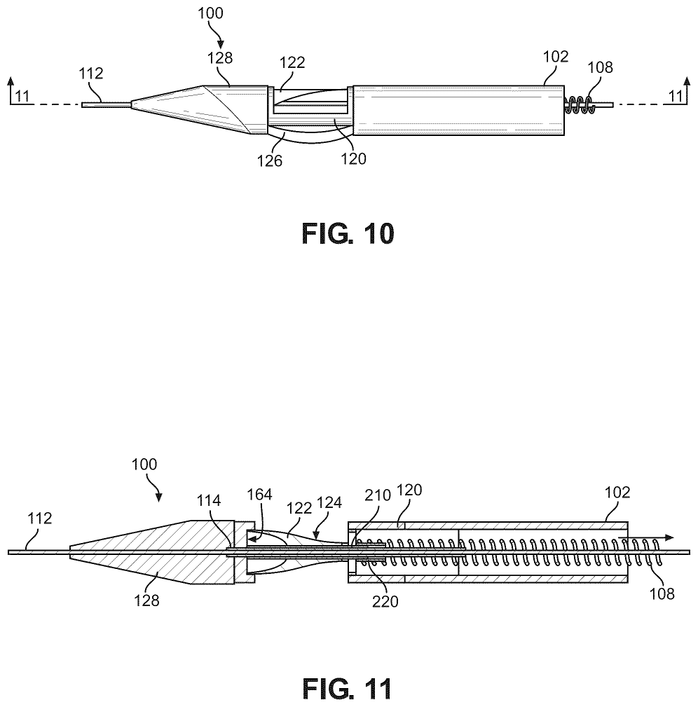

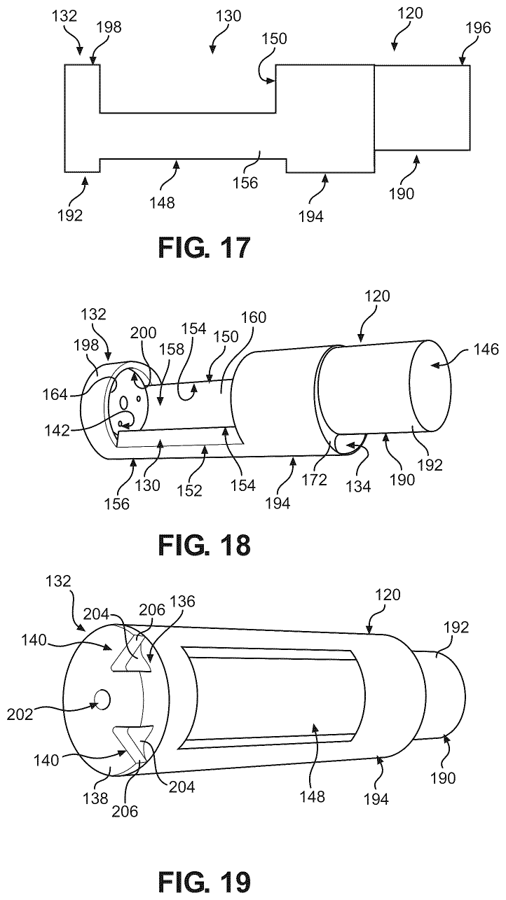

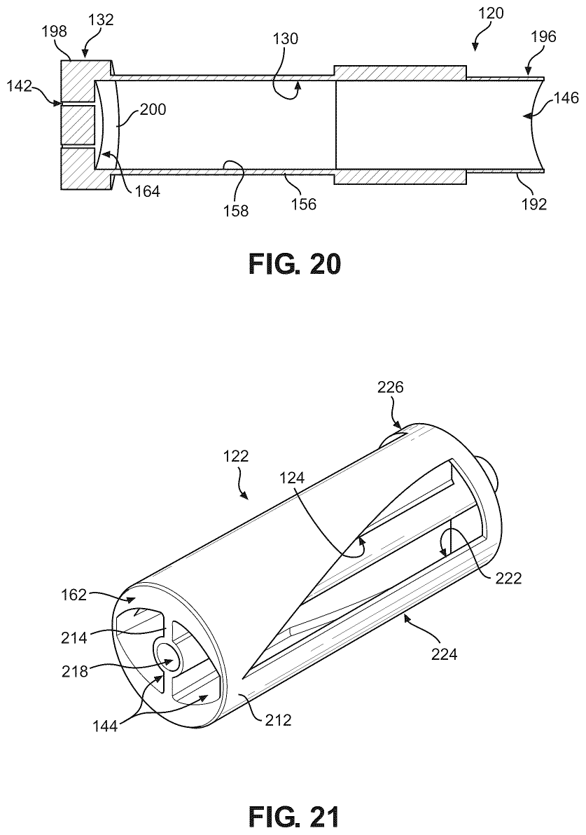

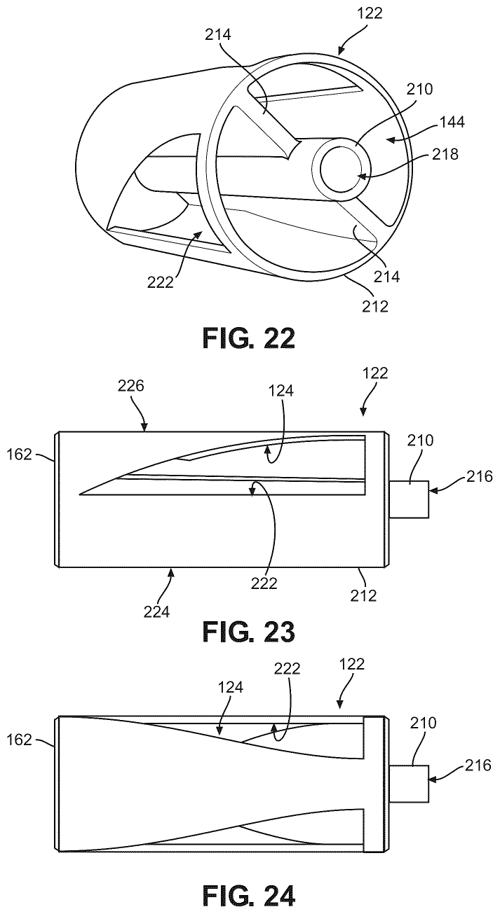

Referring collectively to FIGS. 3-24, the following is a discussion of the various functions of the head 110 of the catheter 100. Once the head 110 is located at the desired target location, the user may choose to initiate, increase, or decrease the fluid flow from the roller pump 14 (by adjusting the flow rate thereof) to simultaneously inflate the balloon 126 and irrigate the cutting chamber 130, rotate the probe 122 to cut the material, oscillate the probe 122 to break up the material via a corresponding resonant frequency (e.g., specific to a type of material and the oscillation speed and/or impact force of the probe 122, generating a resonance effect), and/or oscillate the probe 122 (e.g., adjusting the oscillation speed, i.e., loading speed, and/or impact force, i.e., amplitude, of the probe 122) and/or oscillate the probe 122 at a non-resonant frequency, which can be much lower than a resonant frequency, to pulverize or deform the dislodged material within the cutting chamber 130 (e.g., an impact effect, which may be after the removal thereof from the body lumen). Thereby, the probe 122 may be configured to perform a multitude of functions including removing the material from the body lumen via rotational cutting, dislodge and/or break up material via resonant frequency disruption, and/or pulverize the material within the cutting chamber 130 via impaction against the housing 120. In one embodiment, some operational parameters, such as fluid inflow dependent balloon 126 inflation and irrigation, may be preset and simultaneously activated during the rotational cutting function or the oscillation impact pulverization function of the head 110 of the catheter 100.

Once the head or operational hub 110 is positioned at the desired location within the body lumen, the user can initiate or increase the fluid flow to the head 110. Alternatively, the fluid flow can be automatically activated upon operating the probe 122. The fluid will travel through the infusion lumen 104 of the catheter tube, into an infusion aperture 134 of the housing 120 (FIG. 18), through the balloon 126, enter a fluid inlet compartment 136 integrally formed into an exterior surface 138 of the distal end 132 of the housing 120 via one or more nozzle channels 140 (FIG. 19), and through respective nozzles 142 into the cutting chamber 130. With a sufficient fluid flow provided by the infusion lumen 104, the nozzles 142 create enough backpressure to inflate the balloon 126. Thereby, the balloon 126 can be configured to automatically inflate and expand upon receiving fluid and irrigating the cutting chamber 130. Each nozzle 142 is defined by a bore or aperture that extends through the distal end 132 of the housing 120, between each nozzle channel 140 and the cutting chamber 130. Each nozzle 142 pressurizes the fluid therethrough so that the fluid may irrigate the cutting chamber 130 and subsequently extract the material out of the head 110. More particularly, the material may exit through an extraction aperture 144 of the probe 122, a corresponding extraction aperture 146 of the housing 120, and into the extraction lumen 106 of the catheter tube 102. Thereafter, the heterogenous solution of saline, blood, and material fragments may be collected in the collection tank 30 of the roller pump 14 and disposed of as needed.

Using the balloon 126, the user can selectively bias the housing 120 (in a direction generally opposite of the balloon force applied to the body lumen), stabilize the head 110, and/or contact and dislodge material with the balloon 126 itself as desired. For example, the balloon 126 upon inflation can move the cutting chamber 130 of the head 110 closer to the material in the body lumen, and thereafter maintain a constant and evenly applied contact force against the material, keeping the cutting chamber 130 against the material while moving the head 110 in the body lumen. In detail, the inflated balloon 126 contacts and acts upon the body lumen (applying a biasing force normal to the interior wall of the body lumen). In turn, the inflated balloon 126 pushes the housing 120, and probe 122 therein, closer to the material (in a direction opposite to the balloon biasing force applied to the body lumen) such that the cutting chamber 130 at least partially surrounds or is at least positioned adjacent to the material (allowing the material to prolapse into or sit flush against the cutting chamber 130). Thereby, in one embodiment, the probe 122 only removes material that is within (or immediately next to) the cutting chamber 130. In other words, the material that is removed is only the material that extends beyond an outer periphery of the housing 120 into the cutting chamber 130 (because the probe 122 does not extend outside of the cutting chamber 130), which accordingly prevents unwanted damage to the body lumen.

Additionally, the cutting chamber 130 can be aligned relative to the body lumen, via the nose 128 and/or balloon 126, to accordingly provide for parallel surface-to-surface contact between the head 110 and the body lumen (and material thereon). Therewith, the generally planar (e.g., flat) surface of the head 110 can also help align the cutting chamber to be substantially perpendicular to the material. Furthermore, given the dynamic and deformable nature of the inflated balloon 126, the balloon 126 adapts to the profile of the body lumen and ensures consistent, forced contact between the cutting chamber 130 and the material, along the body lumen, throughout the debulking and/or pulverization of the material. Therefore, the balloon 126 is able to adapt to unique contours of the interior wall and obstructive plaque of the body lumen. The conformability of the balloon 126 allows for the contact with the material to be maintained while enabling motion of the catheter 100 (e.g., longitudinal motion along the length of the lesion), with minimal resistance between the balloon 126 and the body lumen. The balloon 126 also prevents unwanted movement or repositioning of the head 110 (e.g., unwanted rotation) from torque or other impact forces when cutting the material. Once located and locked into a desired position, the cutting chamber 130 and probe 122 can selectively excise the material, layer by layer, with the balloon 126 applying continual and/or increased pressure, until the protruding material (which can be localized and dense) is removed and aligns closely with the longitudinal plane of a healthy wall section of the body lumen. Since the catheter 100 makes a parallel surface-to-surface contact with the body lumen, and since the blade rotates only within the bounds of the cutting chamber 130, the probe 122 may safely treat body lumens in noncompressible parts of the body, such as iliac arteries and also within the re-blockage of previously opened vessels using a stent.

In one embodiment, the balloon 126 is connected to, and is at least partially housed within, an open balloon compartment 148 of the housing 120 opposite a cutout 150 of cutting chamber 130. The balloon 126 is fluidly coupled to the infusion aperture of the housing 120 (and the infusion lumen 104 of the catheter tube) at its proximal end and is fluidly coupled to the cutting chamber 130 via the nozzle channels 140 and nozzles 142 at its distal end. The balloon 126 can comprise a highly compliant, elastomeric, polyurethane, polyester, and/or silicone material. In one embodiment, the balloon 126 is a planar sheet of flexible material, such as latex. In another embodiment, the balloon 126 can be an inflatable tube. In one embodiment, the balloon 126 may comprise a semi-cylindrical shape. The balloon 126 can have a length of 5-20 mm, and more preferably 10-15 mm. The balloon 126 can be rigidly attached at each end to the housing 120 via an epoxy. The balloon 126 can be substantially parallel to the housing 120. In some embodiments, the catheter 100 may include two or more balloons. In one embodiment, the balloon 126 may be coated with a hydrophilic film to reduce friction when the balloon 126 is inflated and in contact with the body lumen.

In a cutting procedure, the probe 122 may shear cut the material in tandem with each side wall 152 of the cutting chamber 130 of the housing 120 (see e.g., FIG. 18). In particular, the cutting chamber 130 comprises a pair of side walls 152 with tapered edges that respectively and each define a sharp cutting edge 154 that is configured to shear cut the material in conjunction with the probe 122, upon rotating the probe 122 within the cutting chamber 130. Each side wall 152 tapers inwardly from the outer periphery 156 to the inner periphery 158 of the housing 120, which accordingly spaces the sharp cutting edge 154 of each side wall 152 away from the outer periphery 156 at a distance which is equal to the width of the side wall 152. In other words, the sharp cutting edge 154 of each side wall is located at an inner surface 158 of an interior wall 160 of the housing 120 (FIG. 18) instead of an outer surface 156 of the housing 120. Thereby, the inwardly tapered side walls 152 may help to reduce unwanted damage to the body lumen because the point at which cutting (i.e., shearing) occurs is spaced further inwardly toward the inside of the cutting chamber 130 (and away further away from the body lumen).

The configuration of a bladed probe 122 and flanking cutting edges 154 on the cutting chamber 130 increases the removal rate of the material, increases the protection of the body lumen (with less accidental damage thereto), and decreases the operational time of the surgical procedure. In particular, the cutting efficiency of the catheter 100 is improved because the bladed probe 122 can rotate clockwise and counter clockwise to cut the material at either cutting edge 154 of the cutting chamber 130 (e.g., allowing for multidimensional cutting of hard calcified atherosclerotic lesions). Additionally, the diagonal orientation of each curved blade 124 of the probe 122 facilitates circumferential and longitudinal slicing of the material, during the rapid rotation of the probe 122. In an example, the cutting process may begin at the lower leading corner of the cutting chamber 130, where the blade of the probe 122 initiates the incision. As the blade of the probe 122 continues to rotate, the initial cut in the material progresses, and concludes at the upper trailing corner of the cutting chamber 130. At this point, a material fragment is fully severed and separated from the body lumen. The angle of the blade of the probe 122 (along with the cutting edges of the cutting chamber 130) and the nozzles 142 can help prevent material fragments from entering the body lumen. For instance, the angle of the blade of the probe 122 ensures that the cut material is directed into the cutting chamber 130 and toward the extraction lumen 106.

The nozzles 142 and/or the roller pump 14 can aspirate the head. In an example, the suctioning force of the roller pump 14 can be strong enough to aspirate the head 110 via the extraction lumen 106. In another example, the nozzles 142 spray fluid at a central location within the cutting chamber 130 and toward the direction of the extraction lumen 106. The high-velocity fluid streams emitted by the nozzles 142 forcibly act on the material fragments, pushing the material fragments toward the extraction lumen 106. Additionally, the high-velocity fluid flow within the cutting chamber 130 (created by the nozzles) generates a venturi effect that helps to suction the material fragments inwardly into the cutting chamber 130 (so that the material is not expelled out of the cutting chamber 130 and into the body lumen). In detail, the nozzles 142 generate a relatively faster flow of fluid through the cutting chamber 130, and thus a lower pressure within the cutting chamber 130 of the housing 120 in comparison to a higher pressure in a surrounding fluid flow of the body lumen, which aspirates the material into the cutting chamber 130. In other words, the blood and/or material fragments in the higher pressure body lumen will flow into the lower pressure cutting chamber 130, further preventing dislodged material from entering the fluid flow of the body lumen. Hence, the suction force (from the roller pump 14 and/or nozzles) may aspirate small amounts of blood from any gaps between the interior wall of the body lumen and the cutout 150 of the cutting chamber 130, thereby further assisting in the retrieval of material fragments or debris. Therefore, the catheter 100 may ensure the capture of any pulverized or cut debris (or fragments) that may have scattered outside the cutting chamber 130.

In a material deforming and/or removing procedure (i.e., pulverization procedure) via resonant frequency, the probe 122 may linearly translate or oscillate back and forth (i.e., vibrate), at a predetermined speed, and forcibly contact the housing 120 (e.g., any inside surface of the cutting chamber 130). Oscillating the probe 122 generates vibrations within the probe 122 and/or housing 120. Such vibration emits a desired frequency (emanating from the probe 122 and/or housing 120), such as a natural frequency of plaque, e.g., 20-500 Hz, to accordingly cause the material (e.g., plaque) to vibrate at a corresponding resonant frequency, thereby dislodging, deforming, breaking up, and/or otherwise weakening the material. In one embodiment, the linearly oscillating the probe 122 only deforms the material, preparing the material for subsequent shear cutting from the body lumen.

The oscillation speed, impact frequency, and/or impact force (generating resulting impact energy) can be adjusted to target a specific type of material. In an example, the oscillation of the probe 122 causes the probe 122 to vibrate at a frequency close to a natural frequency (e.g., resonant frequency) of the calcified material, which in turn causes the material to vibrate at corresponding resonant frequency. Thereby, the material may accordingly exhibit peak displacement response and energy response (resonance effect), wherein the material is subjected to both normal and shear stresses that lead to crack formation, crack propagation deeper into the large plaque further away from a point of impact, and eventual breaking up, dislodging, and/or weakening of the material. The oscillation speed (i.e., loading speed), impact frequency, and/or impact force (i.e., amplitude), which generates the resulting impact energy, can be adjusted to target a specific type of material.

In one embodiment, the probe 122 and/or housing 120 may emit a frequency of 10-100 kHz, and preferably 20-50 kHz to target specific types of heterogeneous plaque. In one embodiment, the impact amplitude of the probe 122 can be 3-5 millimeters. In one embodiment, the frequency, loading speed, and/or amplitude can be adjusted to target specific types of heterogenous plaque. In one embodiment, the user may select or input a type of material via the control unit 16, and the control unit 16 may automatically generate the requisite frequency keyed to the particular type of material.

In a pulverization procedure, via impact effect, the probe 122 can linearly translate back and forth (e.g., vibrate) to forcibly contact and crush the material within the housing 120, to make each piece of material smaller and easier to extract. In an example, the probe 122 can oscillate at a frequency that is less than a resonant frequency or natural frequency of the material to make large calcified plaque material into smaller pieces connected by fibrous tissue, deforming and weakening the large plaque making the plaque vulnerable for excision and removal and/or balloon (or stent) expansion. In one embodiment, the frequency of the vibration impact can be less than 20 kHz, and the amplitude of the impact can be 4-8 millimeters. In operation, after being cut, the material falls further into the cutting chamber 130, wherein the material can be sequentially pulverized and/or excised by the probe 122 crushing the material against the housing 120, i.e., a distal end 132 of the cutting chamber 130 (which is also the distal end 132 of the housing 120). In detail, during an oscillation cycle, as the probe 122 retracts proximally (away from the nose 128) the cut material may fall further inwardly into the cutting chamber 130 and into a gap between a distal end wall 162 of the probe 122 and a bottom wall 164 of the cutting chamber 130. When the probe 122 extends distally (toward the nose 128), the end wall 162 of the probe 122, which acts as an anvil, crushes the material against the bottom wall 164 of the cutting chamber 130. Also, the material can be further pulverized by resonant frequencies caused by vibrations in the probe 122 and/or housing 120, due to the probe 122 oscillating and hitting against the cutting chamber 130. The end wall 162 of the probe 122 is substantially parallel to and faces the bottom wall of the cutting chamber 130, when the probe 122 is seated within the cutting chamber 130. The end wall of the probe 122 can be a flat or angled wall. In other words, the juxtaposed surfaces of the probe 122 and the cutting chamber 130 may respectively define pulverization surfaces for crushing the material therebetween. After pulverization (or fragmentation), the pulverized material exits internally through the probe 122, the housing 120, and the extraction lumen 106. Pulverizing the material may help improve the overall safety of the surgical procedure because the pulverized hard, calcified and large lesion has been modified to allow for excision, removal, and confirmable for balloon expansion as opposed to a non-pulverized large and calcified lesion penetrating the outer wall with radial balloon expansion, causing vessel damage, e.g., rupture or perforation. Moreover, if any pieces of pulverized material escape the housing 120 such pulverized pieces may be too small to cause downstream obstruction in the vessel or other undesired consequences which could have resulted from relatively large pieces of material in the vessel.

The user can select any of the aforementioned functionalities of the catheter 100, in isolation or in tandem with one another. For example, if the user chooses to oscillate, and rotate, the probe 122 within the housing 120, the probe 122 can collectively cut material from the body lumen, pulsate at a desired resonant frequency to modify the material (e.g., break up, deform, etc.), pulverize the material by crushing the material against an interior surface of the cutting chamber 130, cut or dislodge the material, and thereafter extract the cut and pulverized material out through the extraction aperture 144 of the probe 122 and the corresponding extraction aperture 146 of the housing 120.

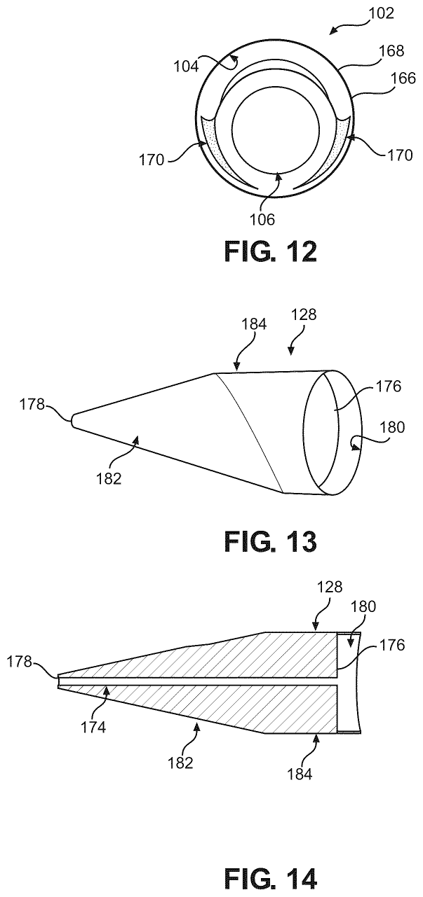

Referring specifically to FIG. 12, in one embodiment, the catheter tube 102 comprises a multi-lumen catheter tube 102 with an annular outer wall 166 and an annular inner (or intermediary) wall 168 within the outer wall 166, creating an outer, infusion lumen 104 and an inner, extraction lumen 106 (or retrieval lumen), separated and spaced apart from one another by the inner wall 168. In other words, the catheter tube 102 may comprise two or more internal lumens 104, 106 for the infusion and extraction of fluid, respectively. The infusion lumen 104 is configured to supply fluid to the head 110 (for inflation of the balloon 126 and irrigation of the cutting chamber 130). The infusion lumen 104 extends from the infusion port 24 to the infusion aperture 134 of the housing 120, next to the proximal end of the balloon 126. The inner wall 168 is shorter than the outer wall 166 such that a distal end 170 of the inner wall 168 defines arcuate mating surfaces 170 that mate with corresponding arcuate mating surfaces 172 of the housing 120 (FIG. 18) when the housing 120 is seated within the catheter tube 102.

In one embodiment, the infusion lumen 104 can have an ellipsoidal or crescent shaped cross-section, and the extraction lumen 106 can have a circular cross-section. In one embodiment, the cross-sectional shape of the infusion lumen 104 can be oblong to generate additional fluid pressure and decrease an overall profile of the catheter tube. The oblong shape of the infusion lumen 104 maximizes the cross-sectional area of the infusion lumen 104, increases the effective radius and reduces the resistance for infusion of fluid at an ideal flow rate, and minimizes the overall diameter of the catheter tube. The infusion and extraction lumens 104, 106 can differ in size or be substantially equal to one another. In one embodiment, the extraction lumen 106 can be greater in size than the infusion lumen 104. The catheter tube can be 50-150 cm in length. The catheter tube 102 (e.g., the infusion lumen 104, extraction lumen 106, inner wall, and/or outer wall) may comprise a flexible polymer material that is embedded metal braiding and/or a fiber mesh and/or coated with a film. The extraction lumen 106 is configured to extract the cut material fragments and aspirate (i.e., suction) the cutting chamber 130 under negative pressure. The extraction lumen 106 extends from the extraction port to the extraction aperture of the housing 120. In one embodiment, the extraction lumen 106 can operate at a higher flow rate compared to the flow rate of the infusion lumen 104. The simultaneous irrigation and extraction of the material fragments, via the bidirectional fluid flow from the infusion lumen 104 to the extraction lumen 106, may also allow for longer, uninterrupted operational run times, in comparison to other catheters. In one embodiment, a diameter of the extraction lumen 106 can be slightly larger than a diameter of the cutting chamber 130.

Referring specifically to FIGS. 13-14, in one embodiment, the nose 128 comprises a solid body cone 128 with a wire bore or aperture 174 extending therethrough from a proximal end 176 to a distal end 178. The wire aperture 174 is configured to receive the guide wire 112 therethrough for easily guiding the head 110 to a desired location within the body lumen. The proximal end 176 of the nose 128 is configured to mate against the distal end 132 of the housing 120. The proximal end 176 of the nose 128 can define a vertical end wall 176 that is substantially flat and which faces and rests flush against the distal end 132 of the housing 120. In one embodiment, a perimeter wall or lip 180 extends outwardly from the proximal end 176 and surrounds the distal end 132 of the housing 120 when assembled, further securing the nose 128 to the housing 120. The distal end 178 of the nose 128, opposite the proximal end 176, defines a distal apex 178 of the nose 128. The nose 128 can be coaxially connected to and coplanar with the distal end 132 of the housing 120. In one embodiment, the nose 128 can be rigidly attached to the housing 120 via an epoxy, ultrasonic welding, and/or compression fit. The nose 128 can comprise a plastic or metal material, which may be embedded with metal braiding for added strength. In one embodiment, the metal braiding of the nose 128 can be welded to the housing 120 to rigidly secure the nose 128 onto the housing 120.

The nose 128 can have a multiplanar surface profile. For example, in one embodiment, the nose 128 comprises a first section 182 with a first angle and a second section 184 with a second angle, that differs from the first angle, to assist in guiding the head 110. The longer first conical section 182 may help rotate the nose 128 upon contacting the material, and the shorter second cylindrical section 184 may help level out the nose 128, and the cutting chamber 130 of the housing 120 therewith, so that the cutting chamber 130 is substantially parallel to the body lumen. In one embodiment, the first conical section 182 can have a length of 10-30 mm, and the second cylindrical section 184 can have a length of 5-10 mm.

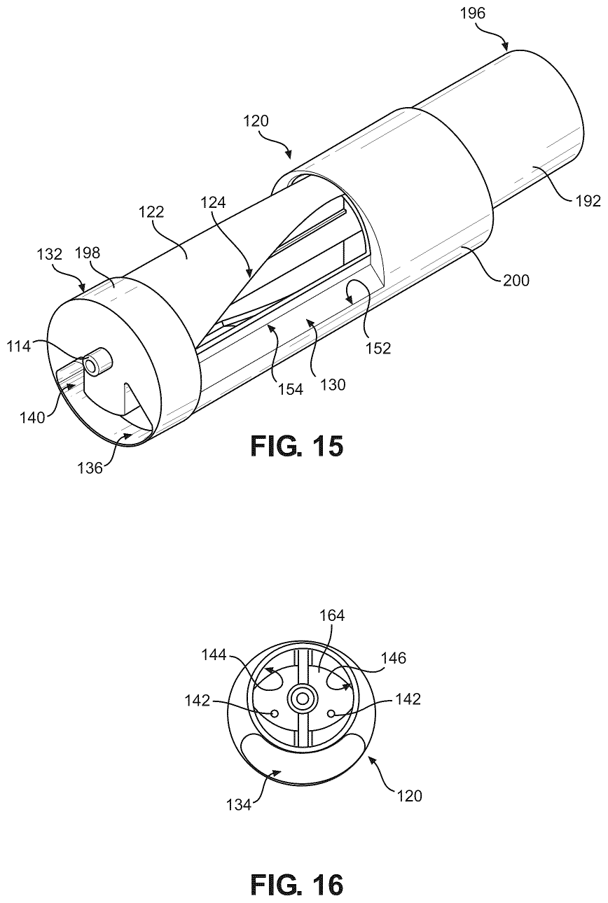

Referring specifically to FIGS. 15-20, in one embodiment, the housing 120 comprises a two-part shell body with a first annular section 190 defining a mounting section 192 which mounts the catheter tube 102, and a second annular section 194 that is offset from the first annular section 190. The first annular section 190 comprises a through bore that defines the extraction aperture 146 and the internal void or bore of the cutting chamber 130. The first annular section 190 also receives the probe 122 therein and allows the probe 122 to oscillate back and forth between the extraction aperture 146 and the cutting chamber 130. The second annular section 194 defines the bounds of the cutting chamber 130, the infusion aperture 134, and the balloon compartment 148. The first and second annular sections 190, 194 are integrally formed with one another. The first annular section 190 can have a smaller diameter than the second annular section 194. A proximal end 196 of the housing 120, defined by the first section 190, is connected to the catheter tube 102, and the distal end 132 of the housing 120 mounts the nose 128. In one embodiment, the housing 120 is comprised of metal, such as stainless steel. In another embodiment, the housing 120 is comprised of a plastic material and the edges of the cutting chamber 130 comprise metal blades. The cutting chamber 130 and the open balloon compartment 148 can be located at opposite sides of the housing 120.

In one embodiment, one or more edges of the cutting chamber 130 can be rounded or beveled to reduce friction for easily moving the catheter 100 through the body lumen. The cutting chamber 130 can be formed by a curved (e.g., concaved) wall 160 of the housing (which is a portion of the second annular section 194). The distal end or base 132 of the cutting chamber 130 (which is the distal end 132 of the housing 120) can comprise an annular disc 198, extending from the curved wall 152, with a rim or lip 200 (FIG. 18). The curved annular disc 198 may have a thickness of 2-3 mm. The lip 200 receives an end portion of the probe 122 when the probe 122 is positioned in its extended position, further securing the probe 122 within the cutting chamber 130. The disc 198 (or distal end 132) can also include a central guide wire bore 202 which receives the guide wire 112 therethrough. The disc 198 can serve as a fixed foundation, absorbing the dynamic impact forces and compressive stress generated when the probe 122 engages the material, e.g., hard calcified plaque, and/or during vibration impact pulverization. Additionally, the disc 198 mitigates a damping effect caused by oscillation, enhancing the overall efficiency of the catheter 100 during operation. The cutting chamber 130 can have a length of 6-12 mm.

In one embodiment, each nozzle channel 140 of the cutting chamber 130 comprises a cutout in the distal end of the housing 120. Therein, each nozzle channel 140 has an open distal end and an open radial side end (unnumbered) next to the balloon compartment 148. Each nozzle channel 140 can define a first wall 204 and a second wall 206 extending outwardly and perpendicularly from the first wall 204 (FIG. 19). Each nozzle channel 140 can be wider at its radial side (next to the balloon compartment 148) and taper inwardly toward the respective nozzle 142 (next to the central axis of the head). When the nose 128 is connected to the housing 120, the proximal end 176 of the nose 128 covers the open distal ends (unnumbered) of the nozzle channels 140, forcing the fluid to flow from the open side (unnumbered) of each nozzle channel 140 toward the respective nozzle 142, effectively redirecting and reversing the fluid flow. Given the taper of each nozzle channel 140, each nozzle channel 140 may serve to guide, direct, and pressurize the fluid toward each respective nozzle 142. Each nozzle 142 may or may not taper to increase the fluid pressure of the fluid which passes therethrough.

Referring to FIGS. 21-24, in one embodiment, the bladed probe 122 comprises a shell body with a central tube 210, and an outer annular wall 212 connected to the central tube 210 by one or more brace members or buttresses 214. The proximal end 216 of the central tube 210 extends outward from and proximally above the outer annular wall 212. The central tube 210 has a guide wire bore 218 for receiving the guide wire 112 therethrough. The proximal end 216 of the central tube 210 can mount a mounting bracket 220 of the flexible drive shaft 108 (FIG. 3), in order to rigidly attach the flexible drive shaft 108 to the probe 122, via an epoxy or fastener. The mounting bracket 220 can comprise a tubular connector that surrounds the proximal end 216 of the central tube 210 of the probe 122. One or more extraction apertures 144 can extend throughout the shell body of the probe 122. For example, in one embodiment, the probe 122 includes left and right extraction apertures 144 at either side of the brace members 214. Each extraction aperture 144 is fluidly coupled to the extraction aperture 146 of the housing 120 such that the pulverized material easily exits internally through the probe 122, the housing 120, and the extraction lumen 106. It is conceivable that the entire bottom surface of the distal end wall 162 of the probe 122, or a portion thereof, may be used to contact and/or crush material against the bottom wall 164 of the cutting chamber 130.

In one embodiment, the probe 122 includes at least one blade 124 with a corresponding blade cutout 222 extending through the outer annular wall 212 (FIG. 21). As shown in FIGS. 21-24, the probe 122 includes a pair of left and right cutting blades 124 with respective blade cutouts 222. Each blade 124 comprises a single, curved cutting edge. One blade 124 can be used for clockwise shear cutting, and the opposite blade 124 can be used for counter clockwise shear cutting. The blades 124 can be disposed on one half of the body of the probe 122, forming a bladeless (i.e., monolithic or probing) section or side 224 opposite a bladed section or side 226 of the body of the probe 122. In one embodiment, the structure of each blade 124 is broader and thicker at the distal end and becomes progressively narrower and lighter toward the proximal end, resembling the shape of an aircraft propeller blade. In one embodiment, both the probing side 224 and cutting side 226 of the operating probe 122 are thicker at the distal base and gradually taper toward the proximal end. Thereby, the mass and weight distribution of the probe 122 may be balanced between the probing and cutting sides 224, 226 to prevent lateral vibrations during rotational operation.

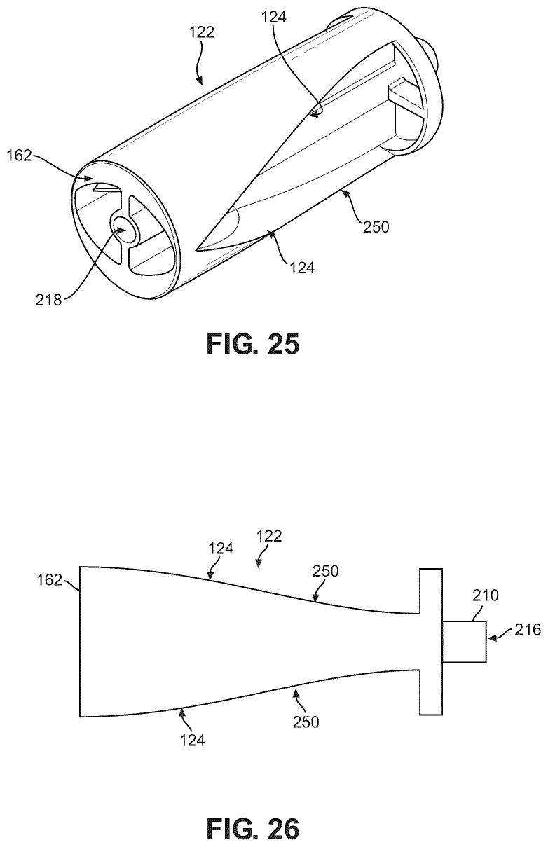

Referring to FIGS. 25-26, in one embodiment, the bladed probe 122 can be in the form a dual-sided probe 122 that includes a pair of dual sided cutters 250. Each cutter 250 can include two obliquely oriented curved blades 124. Thereby, in total, the probe 122 may include four blades 124 for removing a greater amount of material, in comparison to the probe 122 as discussed above. One blade 124 is oriented clockwise, while the other is oriented counter clockwise. The broader distal end of the operating probe 122 may also serve as a vibrational impact probe, as discussed above. The probe 122 can have a diameter of 1.5-1.8 mm.

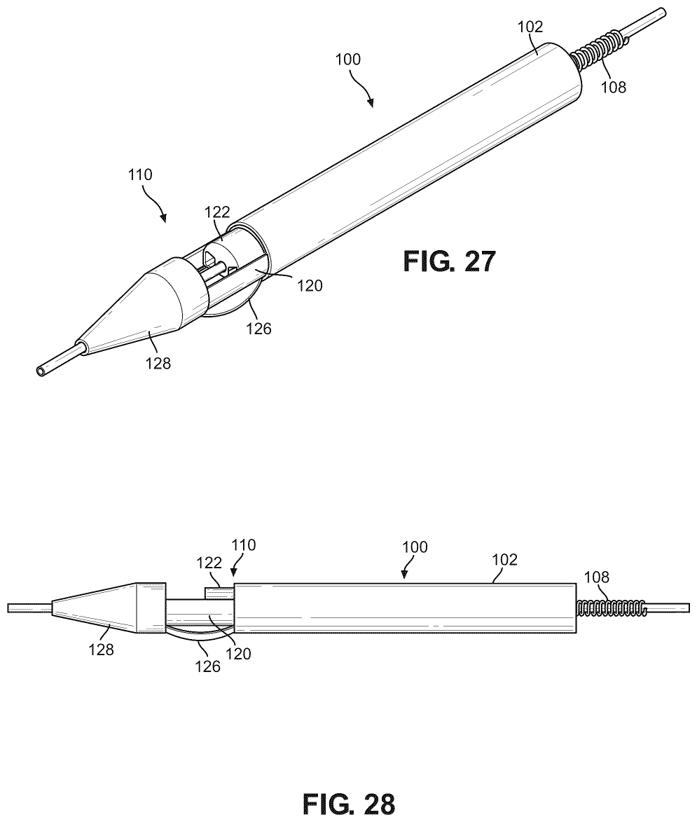

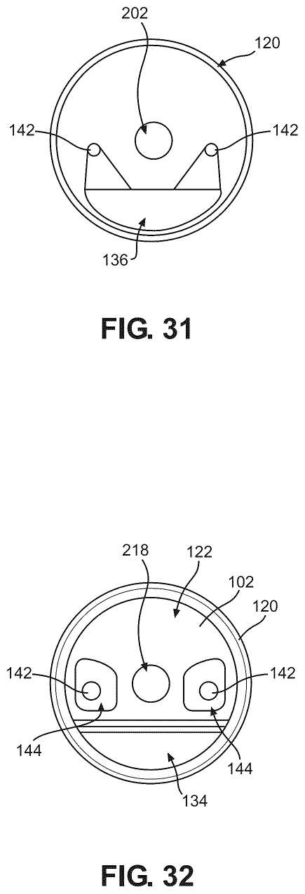

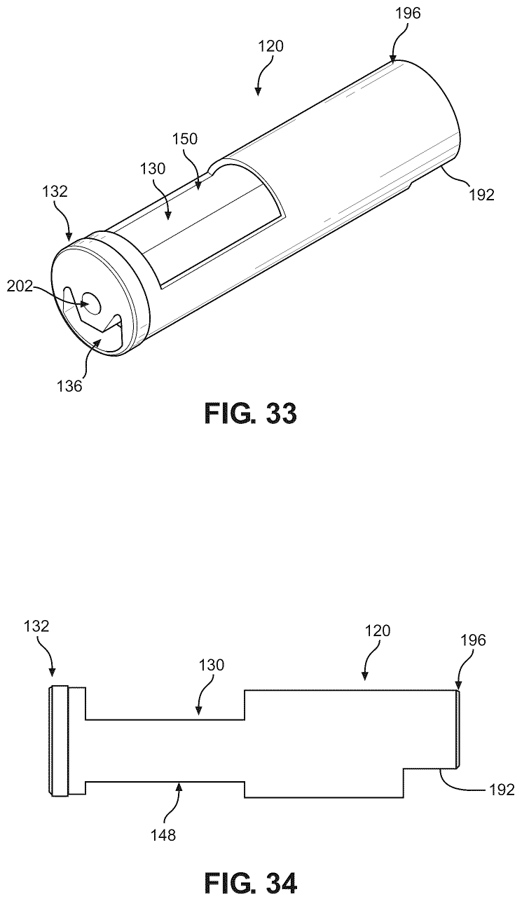

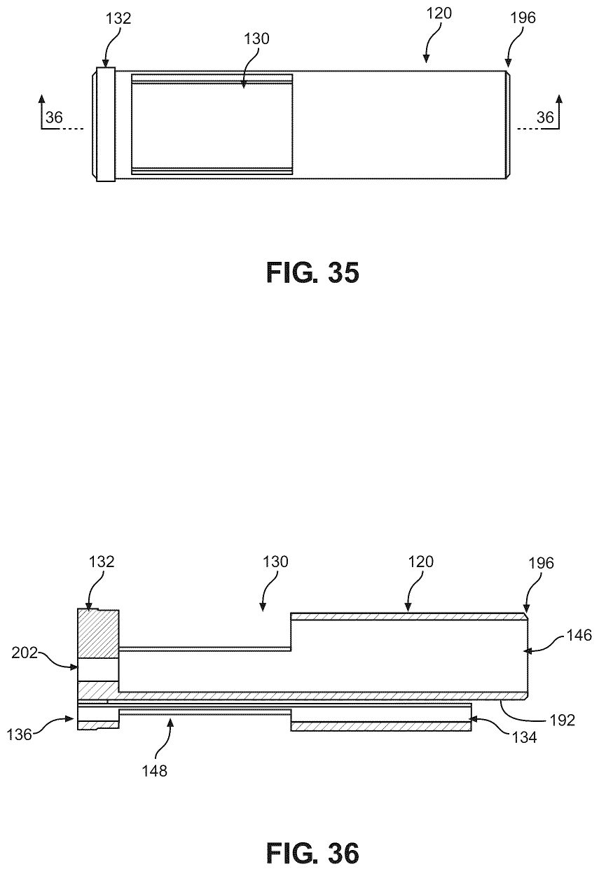

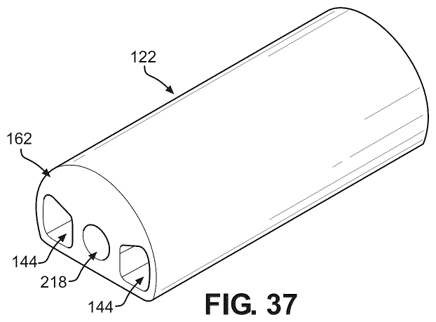

Referring to FIGS. 27-37, there is shown another exemplary embodiment of a catheter 100 which can be substantially similar to the catheter 100 as described above, except that the catheter 100 is configured as a non-cutting or bladeless catheter. Additionally, in one embodiment, the catheter 100 can be smaller than the catheter 100 as discussed above, and thereby usable in smaller body lumens. Despite the absence of the rotational cutting function, the catheter 100 retains its dual-lumen irrigation system, which supports simultaneous plaque fragment retrieval, aspiration, and dynamic balloon-assisted catheter engagement. Like elements have been identified with like reference characters.

The probe 122 of the catheter 100 may only remove and/or pulverize material via vibratory impact or oscillation. The probe 122 may comprise one or more extraction apertures 144 which extend through the body of the probe 122. In one embodiment, the probe 122 comprises a pair of opposed and mirrored extraction apertures 144, flanking a guide wire aperture 218. The probe 122 and the cutting chamber 130 may correspond in shape and size. Therein, the probe 122 may comprise a semicircular cross-section with a flat bottom that mates with and slides against a flat bottom surface of the cutting chamber 130.

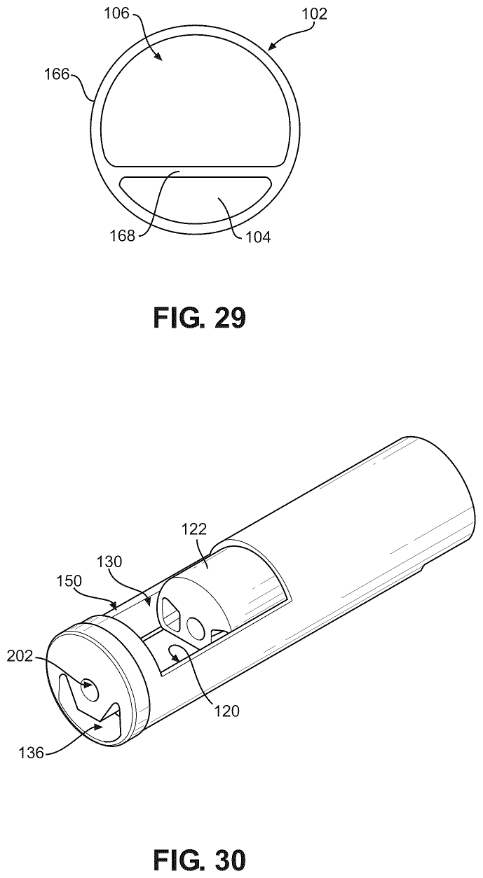

Referring specifically to FIG. 29, the catheter tube 102 of the catheter 100 can comprise a reduced profile, dual-lumen catheter tube 102. The catheter tube 102 comprises an outer wall 166 and a planar inner wall 168 that separates and defines the infusion and extraction lumens 104, 106. In one embodiment, the planner inner wall 168 is located offset from the center of a circular catheter, creating an unequal size semicircular lumen. As shown, the extraction lumen 106 is larger than the infusion lumen 104. In one embodiment, vibration impact may be predominantly for deforming the plaque. In another embodiment, vibration impact may only be for deforming the plaque.

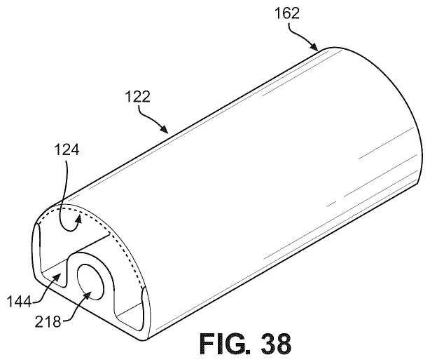

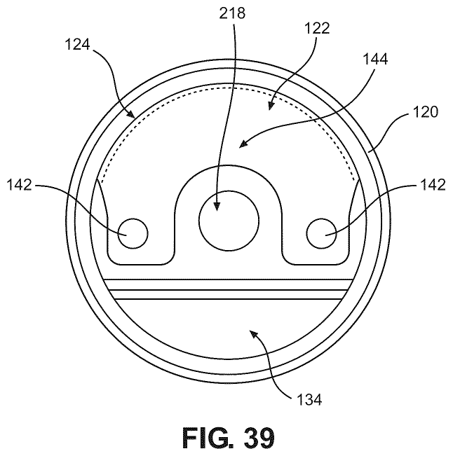

Referring to FIGS. 38-39, there is shown another exemplary embodiment of a bladed probe 122. The bladed probe 122 can be used in a relatively smaller sized catheter 100 or a larger sized catheter 100, as discussed above. The probe 122 includes a blade 124 and a single enlarged extraction lumen 144. The blade 124 is located at a distal end of the body of the probe 122. As opposed to a rotating blade that shear cuts the material, the blade 124 is configured as a linear-end blade for linearly cutting the material only during oscillation of the probe 122. The blade 124 may also cut or crush the material against the juxtaposed wall of the cutting chamber 130. The blade 124 can be mounted to an end wall and/or an interior wall of the probe 122. The blade 124 can comprise one or more blade segments. The blade 124 can be oriented parallel, perpendicularly, or obliquely relative to a longitudinal axis of the probe 122. To accommodate the enlarged extraction lumen 144, the probe 122 has a reduced profile end wall 162. In operation, the end wall 162 can contact the housing 120 for pulverizing the material. In an alternative embodiment, the blade 124 may be disposed at the outer periphery of the probe 122. In one embodiment, the probe 122 and/or the cutting chamber 130 may include one or more mechanical stops, such as rubber stoppers or gromets, that prevent the blade 122 from contacting the interior of the cutting chamber 130. In another embodiment, the end wall 162 of the probe 122 may extend further distally of the blade 124, thereby preventing the blade 124 from contacting the interior of the cutting chamber 130. Like elements have been identified with like reference characters.

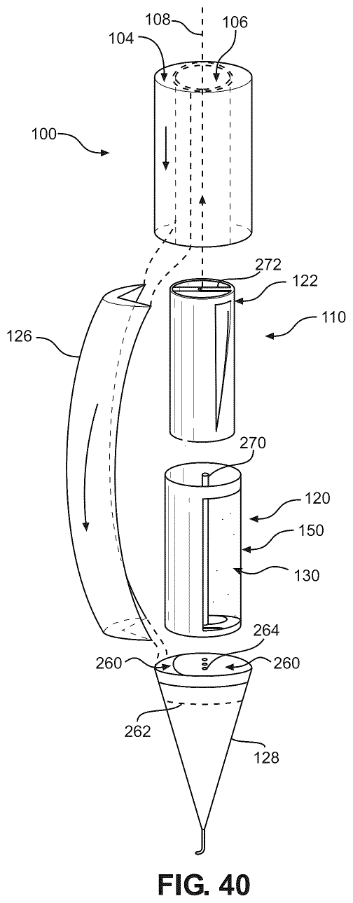

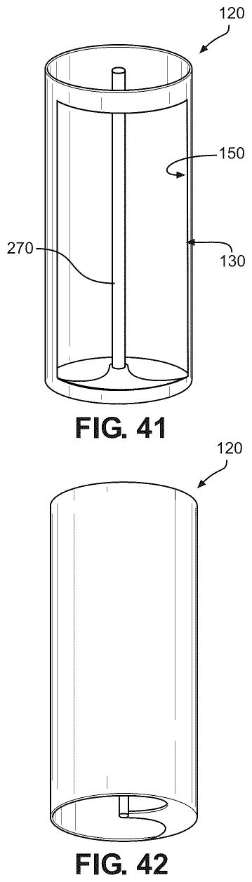

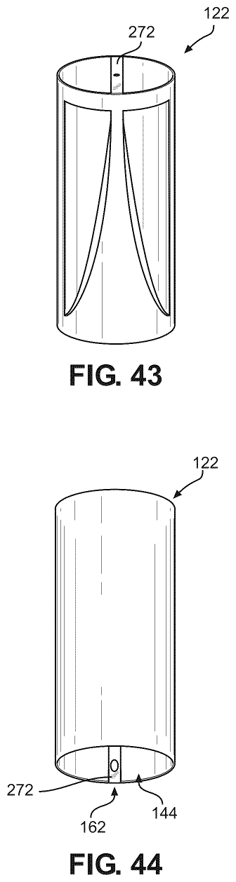

Referring to FIGS. 40-44, there is shown another exemplary embodiment of a catheter 100 which can be substantially similar to the catheters 100 as described above, except that the catheter 100 includes a tubular housing 120 and a hollow or shell nose 128 for reversing and pressurizing the fluid flow into and through the cutting chamber 130. Like elements have been identified with like reference characters.

The hollow nose 128 is configured to receive the fluid from the balloon 126 and redirect the fluid into the cutting chamber 130. The hollow nose 128 can include a fluid inlet 260 fluidly connected to the balloon 126, an internal fluid chamber 262 fluidly connected to the fluid inlet 260, and at least one nozzle 264 fluidly connected to the internal fluid chamber 262 and opening toward the cutting chamber 130 of the housing 120. The at least one nozzle 264 is configured to pressurize the fluid passing therethrough such that the fluid flushes out the cutting chamber 130 and forces the pulverized material out through the extraction lumen 106. The at least one nozzle 264 of the hollow nose 128 is further configured to generate backpressure in the internal fluid chamber 262 to inflate the balloon. The balloon 126 comprises a proximal end connected to the infusion lumen 104 and a distal end connected to the fluid inlet 260 of the nose 128.

The tubular housing comprises an open proximal end, defining the extraction aperture 146, and a partially closed distal end 132 that defines a bottom wall 164 of the cutting chamber 130. The bottom wall 164 of the cutting chamber 130 may only extend across a portion of the distal end 132 of the housing 120. The housing 120 can also include a central post or tube 270 extending upwardly from the bottom wall 162. The central tube 270 of the housing 120 mounts one or more mating features 272, e.g., mating tabs, braces, beams, etc., of the probe 122. A distal most mounting feature 272 of the probe 122 may also define the bottom end wall 162 of the probe 122.

The catheters as disclosed herein can be variously sized to accordingly fit within correspondingly sized body lumens and to remove specific types of material therein. The catheters may range in size from small, medium, large, to extra-large. In one embodiment, the catheters may range in size from 0.33 mm to 2.33 mm in diameter. The small catheters, for example, less than 2 mm, may or may not include bladed probes. Therein, a small catheter may only utilize axial vibro-impact to remove and/or pulverize plaque. In one embodiment, the small catheter may comprise a profile of 6 Fr, with an inner diameter of 1.8 to 2 mm. A small catheter may be usable in a body lumen which is 3-5 millimeters in diameter. In an example, a small catheter may be suitable for superficial femoral artery and common femoral interventions. For smaller atherectomy catheters, the ratio of inflow rate to outflow rate can be up to 1:2. For medium or large atherectomy catheters, this ratio can be smaller as the overall catheter size is larger and can accommodate adequate inflow and outflow lumen sizes for reducing the flow resistance and increasing the flow rate without adverse flow restrictions. A medium size catheter, e.g., smaller than 2.33 mm in diameter, may or may not utilize a bladed probe. In one embedment, a medium size catheter may comprise a profile of 7 to 8 Fr, with an inner diameter of 2 to 2.8 mm, and more preferably 2.2 to 2.6. A medium size catheter may be suitable for iliac artery intervention. A larger catheter, e.g., approximately 2.33 mm in diameter, plus or minus 1 mm, may be suitable for intervention involving acute and chronic venous thrombosis, fibrotic venous occlusive disease involving lower extremity femoral popliteal and iliac deep venous system, and lesions involving dialysis AV fistula, AV fistula to venous anastomosis and venous outflow of the graft. In one embodiment, the larger catheter may comprise a profile of 8 to 10 Fr, with an inner diameter of 2 to 3.5, and more preferably 2.6 to 3.2 mm. In one embodiment, a large or extra-large catheter may comprise a profile of 10-16 Fr, with an inner diameter of 3 to 6 mm, and more preferably 3.2 to 5.2 mm.

In an alternative embodiment, the catheter may not include a probe within the housing. In other words, the catheter can be operated without an operating probe such that the cutting chamber functions as an open chamber for capturing material therein. Therefore, the catheter may operate as a suction/aspiration catheter for the removal of intravascular clots, foreign body, and loose plaque material.

In one embodiment, the probe can be assembled into the system as a permanent part of the unit. In another embodiment, the probe can be removable and replaceable or interchangeable with a differing probe as desired. The user can exchange the probes disclosed herein for better removing particular types of lesions. For instance, a cutting probe may be replaced with a pulverizing probe that has pressure points on the distal end thereof for multiple micro-indentation during plaque pulverization, which may be used for densely calcified lesions.

In an alternative embodiment, the balloon of the catheter can be used independently via a designated balloon fluid line. Therein, the catheter may inflate or deflate the balloon independently of irrigation or flushing out the cutting chamber. The catheter can include a closed loop, continuous flow through the balloon lumen to inflate and deflate the independent dynamic balloon. Thereby, the balloon can be separately used to engage lesions or other material. In another alternative embodiment, the catheter can include two balloons. Therein, the catheter may include an internal (intravascular) balloon at the opposite side of the window of the cutting chamber and an external balloon at the proximal end of the catheter outside of the body. Both balloons are connected by an elongated tubing with a side port and locking system for the fixed volume of fluid input to inflate both balloons. As there is no continuous flow through the elongated connecting tube, the resistance from the tube is not a factor between the connected balloons. The volume exchange between the two balloons allows for the internal balloon to adapt to the space available. Since the external balloon is stiffer (less elastic) than the internal balloon, the latter expands more until the external resistance in the lumen limits the expansion and the fluid will flow back into the external balloon. Instead of using an external balloon, several alternative mechanisms can be employed, such as pressure from hydralazine/pneumatic systems or spring-assisted volume displacement methods.

In one embodiment, the catheter system can further include a pressure monitoring system. For example, a pressure monitoring system can be coupled to the catheter tube to continuously monitor fluid pressure during operation of the catheter. The pressure monitoring system may ensure that the fluid pressure in the infusion line and/or head does not exceed a maximum pressure, which may burst the balloon. Additionally, the pressure monitoring system may also monitor negative pressure at the outlet of the outflow lumen during the catheter's aspiration function. High negative pressure readings may indicate a potential blockage in the outlet lumen. The control unit may automatically adjust the roller pump to increase or decrease the fluid pressure in the infusion and/or extraction lumen, based upon the sensed fluid pressure provided by the pressure monitoring system. In one embodiment, the ratio of the flow rates between the extraction and infusion lumens can range from a 1-to-1 ratio to a 2-to-1 ratio, or more if desired during an irrigation function, wherein the extraction lumen operates under a lower pressure than the infusion lumen. In one example, the extraction lumen removes equal output and input volume at a 1-to-1 ratio and twice output to input volume at a 2-to-1 ratio. Additionally, during the aspiration function, the output volume is variable depending on the size of the extraction lumen of different size catheters and the pump output flow rate generate negative suction pressure. The actual flow rates between the extraction and infusion lumens may be dependent on their respective size, which ultimately determine the overall size of the catheter tube.

In one embodiment, the catheter can be used to treat one or more specific and differing types of material within the body lumen. For example, the catheter may remove different types of plaque that have distinct biological compositions and mechanical behaviors. In general, prior art atherectomy devices often achieve suboptimal results for the treatment of hard nodular calcified lesions for several reasons. Firstly, a lesion can be highly resistant and may be deflected. Secondly, the device itself can also be easily displaced and rotated away from the lesion during its operation or the cutter may not come in contact with the lesion, making it difficult to keep the prior art device at one specific location. Thirdly, the cutting mechanism could be less efficient in cutting through hard, calcified anisotropic plaque material. Lastly, when radial force is applied to a large hard calcified lesion, the recoil of the lesion requires placement of a stent to act as a scaffolding in the body lumen. In contrast to such prior art devices, the head of the catheter can remain stationary at a single location, without significantly moving during operation thereof, because the head becomes wedged against the material as a result of the balloon pressing the cutting chamber against the material, which assists in locking the head in position. Furthermore, the probe of the catheter helps to pulverize the material into small pieces. Still further, the irrigation of the cutting chamber helps to remove the material such that a stent may not be necessary. Therefore, the catheter may easily and efficiently remove hard nodular calcified lesions or other material that has been traditionally difficult to remove and breakup. Thus, high-intensity axial (longitudinal) force can be safely applied in a vessel as there is a greater degree of redundancy or laxity in the vascular structures longitudinally. The catheter may be operated using predominantly axial (longitudinal) force while minimizing the radial force.

In one embodiment, the catheter may utilize a myriad of differing functionality to target multiple, differing types of materials. For example, the catheter may utilize five differing mechanisms, including plaque pulverization, plaque excision through oblique and linear cutting, irrigation of the operational hub to remove dislodged material, inflation and deflation of the compliant balloon, and precision delivery of contrast agents, e.g., dye, or another substance, e.g., medication via the infusion lumen. Thereby, the catheter allows for the initial local injection of contrast agents for angiographic imaging. This provides real-time visual guidance of the lesion area, helping operators to assess the anatomy and plan the intervention. During the procedure, periodic angiographic images can be taken to monitor the progress of the atherectomy ensuring the procedure is proceeding as desired. Additionally, after a period of flushing to remove any debris or cut fragments from the catheter, the catheter can also be used to inject medications locally through the catheter's extraction lumen. These medications could be anticoagulants, vasodilators, or other agents aimed at improving the procedural outcome or preventing complications.

In one embodiment, the plaque pulverization mechanism utilizes focused, linear, high frequency, directional impact (between the probe and housing) to pulverize hard calcified nodular plaque. Since heterogeneous hard atherosclerotic lesions are predominantly composed of moderate to heavy calcification plaque tissues, such material can be brittle in nature, and therefore the targeted resonant frequency can assist breaking up the material, increasing plaque removal rates. For example, the mechanism can use axial (longitudinal) high-frequency vibration impact of the cylindrical probe delivered to the calcified nodular lesions that prolapse into the cutting chamber, transmitting vibro-impact force to the plaque. The dynamic load when applied at frequencies close to the natural frequencies of the plaque and with sufficiently high amplitudes can cause the calcified plaque to fracture and pulverize. The elements of the heterogeneous plaque lesion can exhibit various degrees of displacement response and energy response under vibro-impact force, due to variation in their stiffness and damping properties. Also, due to the presence of natural cracks in the plaque, the stress distribution may become extremely uneven at the moment of impact load. The resulting effect is a shear strain between the microelements within the plaque, causing them to separate and fracture. Since the tensile strength of plaque is much smaller than the compressive strength, the tensile stress caused by high frequency dynamic impact load on the plaque may cause continuous damage accumulation. Eventually, when the total damage of the plaque exceeds its tensile fracture or fatigue limit, fragmentation occurs.

The plaque excision mechanism may occur through oblique and linear cutting using the bidirectional rotating cylindrical probe, which operates within the confines of the cutting chamber of the housing, along the plane of contact of the catheter to the interior wall of the body lumen. Such plaque excision can be suitable for the removal of organized thrombus, fibrous plaque, and plaque with mild-to-moderate calcification.

The irrigation mechanism (via the nozzles) is configured to instantaneously remove cut material, e.g., pieces, fragments, particles, etc., during atherectomy. By enabling simultaneous fluid inflow and aspiration outflow through the operating probe and extraction lumen of the catheter, small fragments are efficiently flushed into the collection tank 30 (instead of flowing into the bloodstream of the body lumen). Additionally, in one embodiment, the catheter can function as a stand-alone aspiration catheter by removing the operating probe, disabling the infusion flow side of the pump, and using only the outflow or extraction side of the pump. Aspiration of clots, loose plaque or foreign bodies from the vascular lumen are often required during interventional vascular procedures. The dynamic balloon mechanism, at the opposite side of the cutting chamber, can be used for continuous, active engagement of the cutting chamber to the material, e.g., lesion, during device operation. As the operating probe only affects the plaque prolapsed into the cutting chamber, the device needs to be pressed against the lesion for affective atherectomy. The balloon also stabilizes the operating hub, preventing unwanted movement thereof due to frictional forces between the cutting chamber and the material and/or vessel wall (due to the pressure transmuted through the housing as applied by the balloon). The delivery of various substances mechanism can include the precise delivery of contrast dye close to the material, allowing for detailed imaging and better assessment of material and/or body lumen properties. This facilitates the selection of the most suitable atherectomy mechanism or combination of mechanisms and supports the evaluation of procedural progress and successful completion of the atherectomy process.

Various exemplary embodiments are disclosed herein. One exemplary embodiment includes a catheter with a multipurpose operational hub configured to remove material via resonant frequency vibration pulverization, non-resonant pulverization, rotational cutting, and aspiration.

In one exemplary embodiment, disclosed is a catheter for removing material in a body lumen. The catheter includes a catheter tube, a flexible drive shaft disposed within the catheter tube and configured to be moved by a drive, an infusion lumen disposed within the catheter tube and configured to receive and transport a fluid therethrough, an extraction lumen disposed within the catheter tube. The catheter further includes a head configured to remove the material in the body lumen. The head includes a housing connected to the catheter tube and comprising an infusion aperture fluidly coupled to the infusion lumen, an extraction aperture fluidly coupled to the extraction lumen, and a cutting chamber having a cutout therein. The head further includes a balloon connected to the housing opposite the cutout of the cutting chamber and fluidly coupled to the infusion lumen. The balloon is configured to inflate and expand upon receiving the fluid therein. The head further includes a probe connected to and driven by the flexible drive shaft. The probe is disposed within the cutting chamber, and the probe configured to remove the material from the body lumen, at the cutout, and pulverize the material within the cutting chamber.

In a further example, the probe is configured to linearly translate back and forth within the cutting chamber of the housing such that the probe is configured to collectively cut material from the body lumen, pulsate at a resonant frequency to break up the material, pulverize the material by crushing the material against an interior surface of the cutting chamber, and extract the now pulverized material out through the extraction aperture of the housing.

In a further example, the probe includes at least one cutting blade configured to cut the material only within the cutting chamber upon rotating the probe and an extraction aperture which is fluidly coupled to the extraction aperture of the housing such that the pulverized material exits internally through the probe, the housing, and the extraction lumen.

In a further example, the at least one blade of the probe comprises a pair of cutting blades.

In a further example, the at least one blade of the probe comprises four cutting blades.

In a further example, the cutting chamber of the housing comprises a bottom wall. The probe comprises an end wall which is parallel to and faces the bottom wall of cutting chamber when the probe is seated within the cutting chamber. The probe is configured to linearly translate within the cutting chamber such that the end wall of the probe forcibly contacts the bottom wall of the cutting chamber to dually crush the material therebetween and generate pulsations to further pulverize the material within the cutting chamber.

In a further example, the balloon is configured to expand upon inflation such that the balloon contacts the body lumen and moves the housing, causing the material to enter the cutting chamber for subsequent removal thereof by the probe, within the cutting chamber.

In a further example, the fluid is configured to flow through the infusion lumen, through the balloon to inflate the balloon, into and through the cutting chamber to irrigate and flush the material out of the cutting chamber, and into the extraction lumen for subsequent extraction of the pulverized material.

In a further example, the housing further comprises at least one nozzle fluidly coupled to the balloon, the at least one nozzle opens into the cutting chamber, and the at least one nozzle is configured to pressurize the fluid passing therethrough such that the fluid flushes out the cutting chamber and forces the pulverized material out through the extraction lumen.

In a further example, the housing further comprises at least one nozzle channel extending into an end wall of the housing. The at least one nozzle channel is fluidly coupled to the balloon and the at least one nozzle. The at least one nozzle channel is configured to guide the fluid from the balloon to the at least one nozzle.

In a further example, the head is configured to generate a flow of fluid within the cutting chamber that creates a lower pressure within the cutting chamber in comparison to a higher pressure of a surrounding fluid flow of the body lumen, which aspirates the material into the cutting chamber.

In a further example, the housing further comprises an open balloon compartment located opposite the cutting chamber, and the open balloon compartment is configured to receive and house at least a portion of the balloon therein.

In a further example, the cutting chamber is configured to shear cut the material in conjunction with the probe. The cutting chamber comprises a pair of side walls. Each side wall includes a taper defining a sharp cutting edge that is configured to shear cut the material in conjunction with the probe upon rotating the probe within the cutting chamber.

In a further example, the head further comprises a nose connected to a distal end of the housing.

In a further example, the nose includes a proximal end configured to mate against the distal end of the housing, a perimeter wall extending outwardly from the proximal end and configured to surround the distal end of the housing, a distal end opposite the proximal end and defining a distal apex of the nose, and a wire aperture extending through the nose, from the proximal end to the distal end of the nose, and configured to receive a guide wire therethrough.

In a further example, the nose includes a fluid inlet fluidly connected to the balloon, an internal fluid chamber fluidly connected to the fluid inlet, and at least one nozzle fluidly connected to the internal fluid chamber and opening toward the cutting chamber of the housing. The at least one nozzle is configured to pressurize the fluid passing therethrough such that the fluid flushes out the cutting chamber and forces the pulverized material out through the extraction lumen.

In a further example, the balloon comprises a proximal end connected to the infusion lumen and a distal end connected to the fluid inlet of the nose.

In a further example, the catheter further includes a guide wire extending through the catheter tube and the head, and the guide wire is configured to guide the head within the body lumen.

In another exemplary embodiment, disclosed is a head for an atherectomy catheter configured to remove material from a body lumen. The head includes a housing configured to be connected to a catheter tube. The housing includes an infusion aperture configured to receive a fluid, an extraction aperture, and a cutting chamber having a cutout therein. The head further includes a balloon connected to the housing opposite the cutout of the cutting chamber. The balloon is configured to inflate and expand upon receiving the fluid therein. The head further includes a probe disposed within the cutting chamber. The probe is configured to remove the material from the body lumen, at the cutout, and pulverize the material within the cutting chamber.

In another exemplary embodiment, disclosed is a catheter for removing material in a body lumen. The catheter includes a catheter tube including an infusion lumen and an extraction lumen, a flexible drive shaft disposed within the extraction lumen of the catheter tube, and a head configured to remove the material in the body lumen. The head includes a housing connected to the catheter tube and comprising an infusion aperture fluidly coupled to the infusion lumen, an extraction aperture fluidly coupled to the extraction lumen, and a probe compartment having a cutout therein. The head further includes a balloon connected to the housing opposite the probe compartment and fluidly coupled to the infusion lumen. The balloon is configured to inflate and expand upon receiving the fluid therein. The head further includes a probe connected to and driven by the flexible drive shaft. The probe is disposed within the probe compartment. The probe is configured to oscillate within the probe compartment to pulverize the material within the probe compartment.

In another exemplary embodiment, disclosed is a method for removing material from a body lumen. The method includes the steps of providing an atherectomy catheter, positioning the atherectomy catheter within the body lumen, inflating a balloon to bias a head of the catheter toward the material, cutting the material at the cutting window, pulverizing the material via a vibro-impact force at a resonant frequency, and irrigating and extracting the pulverized material. The steps of the method may be performed in any desired sequence. The method may omit one or more steps described herein. Further, the method may include additional steps not described herein.

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

What is claimed:

1. A catheter for removing material in a body lumen, comprising:
a catheter tube;
a flexible drive shaft disposed within the catheter tube and configured to be moved by a drive;
an infusion lumen disposed within the catheter tube and configured to receive and transport a fluid therethrough;
an extraction lumen disposed within the catheter tube; and
a head configured to remove the material in the body lumen and comprising:
a housing connected to the catheter tube and comprising an infusion aperture fluidly coupled to the infusion lumen, an extraction aperture fluidly coupled to the extraction lumen, and a cutting chamber having a cutout therein;
a balloon connected to the housing opposite the cutout of the cutting chamber and fluidly coupled to the infusion lumen, the balloon configured to inflate and expand upon receiving the fluid therein; and
a probe connected to and driven by the flexible drive shaft, the probe disposed within the cutting chamber, and the probe configured to remove the material from the body lumen, at the cutout, and pulverize the material within the cutting chamber.

2. The catheter of claim 1, wherein the probe is configured to linearly translate back and forth within the cutting chamber of the housing such that the probe is configured to collectively cut material from the body lumen, pulsate at a resonant frequency to break up the material, pulverize the material by crushing the material against the cutting chamber, and extract the now pulverized material out through the extraction aperture of the housing.

3. The catheter of claim 1, wherein the probe comprises:
at least one cutting blade configured to cut the material only within the cutting chamber upon rotating the probe; and
an extraction aperture which is fluidly coupled to the extraction aperture of the housing such that the material exits internally through the probe, the housing, and the extraction lumen.

4. The catheter of claim 3, wherein the at least one blade of the probe comprises a pair of cutting blades.

5. The catheter of claim 3, wherein the at least one blade of the probe comprises four cutting blades.

6. The catheter of claim 1, wherein:
the cutting chamber of the housing comprises a bottom wall;
the probe comprises an end wall which is parallel to and faces the bottom wall of cutting chamber when the probe is seated within the cutting chamber; and
the probe is configured to linearly translate within the cutting chamber such that the end wall of the probe forcibly contacts the bottom wall of the cutting chamber to dually crush the material therebetween and generate pulsations to further pulverize the material within the cutting chamber.

7. The catheter of claim 1, wherein the balloon is configured to expand upon inflation such that the balloon contacts the body lumen and moves the housing, causing the material to enter the cutting chamber for subsequent removal thereof by the probe, within the cutting chamber.

8. The catheter of claim 1, wherein the fluid is configured to flow through the infusion lumen, through the balloon to inflate the balloon, into and through the cutting chamber to irrigate and flush the material out of the cutting chamber, and into the extraction lumen for subsequent extraction of the material.

9. The catheter of claim 8, wherein the housing further comprises at least one nozzle fluidly coupled to the balloon, the at least one nozzle opens into the cutting chamber, and the at least one nozzle is configured to pressurize the fluid passing therethrough such that the fluid flushes out the cutting chamber and forces the material out through the extraction lumen.

10. The catheter of claim 9, wherein the housing further comprises at least one nozzle channel extending into an end wall of the housing, the at least one nozzle channel is fluidly coupled to the balloon and the at least one nozzle, and the at least one nozzle channel is configured to guide the fluid from the balloon to the at least one nozzle.

11. The catheter of claim 1, wherein the head is configured to generate a flow of fluid within the cutting chamber that creates a lower pressure within the cutting chamber in comparison to a higher pressure of a surrounding fluid flow of the body lumen, which aspirates the material into the cutting chamber.

12. The catheter of claim 1, wherein the housing further comprises an open balloon compartment located opposite the cutting chamber, and the open balloon compartment is configured to receive and house at least a portion of the balloon therein.

13. The catheter of claim 1, wherein:
the cutting chamber is configured to shear cut the material in conjunction with the probe; and
the cutting chamber comprises a pair of side walls, each side wall comprising a taper defining a sharp cutting edge that is configured to shear cut the material in conjunction with the probe upon rotating the probe within the cutting chamber.

14. The catheter of claim 1, wherein the head further comprises a nose connected to a distal end of the housing.

15. The catheter of claim 14, wherein the nose comprises:
a proximal end configured to mate against the distal end of the housing;
a perimeter wall extending outwardly from the proximal end and configured to surround the distal end of the housing;
a distal end opposite the proximal end and defining a distal apex of the nose; and
a wire aperture extending through the nose, from the proximal end to the distal end of the nose, and configured to receive a guide wire therethrough.

16. The catheter of claim 14, wherein the nose comprises:
a fluid inlet fluidly connected to the balloon;
an internal fluid chamber fluidly connected to the fluid inlet; and
at least one nozzle fluidly connected to the internal fluid chamber and opening toward the cutting chamber of the housing, and the at least one nozzle is configured to pressurize the fluid passing therethrough such that the fluid flushes out the cutting chamber and forces the material out through the extraction lumen.

17. The catheter of claim 16, wherein the balloon comprises a proximal end connected to the infusion lumen and a distal end connected to the fluid inlet of the nose.

18. The catheter of claim 1, further comprising a guide wire extending through the catheter tube and the head, and the guide wire is configured to guide the head within the body lumen.

19. A head for an atherectomy catheter configured to remove material from a body lumen, comprising:
a housing configured to be connected to a catheter tube, the housing comprising an infusion aperture configured to receive a fluid, an extraction aperture, and a cutting chamber having a cutout therein;
a balloon connected to the housing opposite the cutout of the cutting chamber, and the balloon configured to inflate and expand upon receiving the fluid therein; and
a probe disposed within the cutting chamber, and the probe configured to remove the material from the body lumen, at the cutout, and pulverize the material within the cutting chamber.

20. A catheter for removing material in a body lumen, comprising:
a catheter tube including an infusion lumen and an extraction lumen;
a flexible drive shaft disposed within the extraction lumen of the catheter tube; and
a head configured to remove the material in the body lumen and comprising:
a housing connected to the catheter tube and comprising an infusion aperture fluidly coupled to the infusion lumen, an extraction aperture fluidly coupled to the extraction lumen, and a probe compartment having a cutout therein;
a balloon connected to the housing opposite the probe compartment and fluidly coupled to the infusion lumen, the balloon configured to inflate and expand upon receiving the fluid therein; and
a probe connected to and driven by the flexible drive shaft, the probe disposed within the probe compartment, and the probe configured to oscillate within the probe compartment to pulverize the material within the probe compartment.
